(19) United States
(12) Patent Application Publication
(10) Pub. No.: US 2025/0055318 A1
(43) Pub. Date: Feb. 13, 2025

(54) WEARABLE RING DEVICE FOR ON-THE-GO CHARGING

(71) Applicant: Oura Health Oy, Oulu (FI)

(72) Inventors: Manbir Sodhia, Fremont, CA (US); Kirsi Marja Maansaari, Oulu (FI); Mika Henrikki Törmälä, Oulu (FI); Jussi Petteri Järvelä, Kempele (FI); Jukka Tapani Mäkinen, Oulu (FI)

(21) Appl. No.: 18/447,067

(22) Filed: Aug. 9, 2023

(51) Int. Cl. H02J 50/10; A61B 5/00; H02J 7/00; H02J 50/30; H02J 50/40; H02J 50/90

(52) U.S. Cl. CPC H02J 50/10; A61B 5/6802; H02J 7/0042; H02J 50/30; H02J 50/402; H02J 50/90; A61B 5/6826; A61B 2560/0219

(57) ABSTRACT

Methods, systems, and devices for a wearable device are described. The wearable device may comprise a housing with an inner surface and an outer surface, a battery positioned at least partially within the housing, one or more sensors positioned at least partially within the housing and electrically coupled with the battery, and a wireless charging component disposed at least partially within the housing. The wireless charging component may be configured to perform an inductive charging procedure, a contact-based charging procedure, a photovoltaic charging procedure, or any combination thereof, to wirelessly transfer energy from an external power source to the battery through the outer or inner surface of the wearable device while the wearable device is being worn by the user.

### FIELD OF TECHNOLOGY

The following relates to wearable devices and data processing, including a wearable ring device for on-the-go charging.

### BACKGROUND

Some wearable devices may be configured to collect data from users to help the users understand more about their overall physiological health and well-being. However, wearable devices are only able to collect physiological data while the devices are being worn, and typically have to be taken off and placed on a dedicated charger in order to recharge the devices. As such, conventional wearable devices are unable to collect physiological data while the wearable devices are charging, resulting in gaps in the collected physiological data.

### BRIEF DESCRIPTION OF THE DRAWINGS

FIG. 1 illustrates an example of a system that supports a wearable ring device for on-the-go charging in accordance with aspects of the present disclosure.

FIG. 2 illustrates an example of a system that supports a wearable ring device for on-the-go charging in accordance with aspects of the present disclosure.

FIG. 3 shows an example of a system that supports a wearable ring device for on-the-go charging in accordance with aspects of the present disclosure.

FIG. 4 shows an example of a system that supports a wearable ring device for on-the-go charging in accordance with aspects of the present disclosure.

FIG. 5 shows an example of a system that supports a wearable ring device for on-the-go charging in accordance with aspects of the present disclosure.

FIG. 6 shows an example of a timing diagram that supports a wearable ring device for on-the-go charging in accordance with aspects of the present disclosure.

FIG. 7 shows a block diagram of an apparatus that supports a wearable ring device for on-the-go charging in accordance with aspects of the present disclosure.

FIG. 8 shows a block diagram of a wearable device manager that supports a wearable ring device for on-the-go charging in accordance with aspects of the present disclosure.

FIG. 9 shows a diagram of a system including a device that supports a wearable ring device for on-the-go charging in accordance with aspects of the present disclosure.

FIG. 10 shows a flowchart illustrating methods that support wearable ring device for on-the-go charging in accordance with aspects of the present disclosure.

### DETAILED DESCRIPTION

Wearable devices may be configured to collect physiological data from users to help the users understand more about their overall physiological health and well-being. However, wearable devices are only able to collect physiological data while the devices are being worn, and typically have to be taken off and placed on a dedicated charger in order to recharge the devices. As such, conventional wearable devices are unable to collect physiological data while the wearable devices are charging, resulting in gaps in the collected physiological data. These gaps in collected data may prevent the user from gaining a full picture of their overall health, and may prevent a wearable device from detecting important physiological events for a user that occur when the wearable device is charging.

Accordingly, techniques described herein may support charging of a wearable device, such as a wearable ring device, while the wearable device is being worn by a user. In particular, the wearable device may support charging mechanisms (e.g., electrical contacts and/or inductive charging components) disposed within an outer surface (and/or inner surface) of the wearable device such that the wearable device may charge while the wearable device is being worn, and when the wearable device is positioned next to charging elements positioned on or within everyday objects. For example, phone cases may be built with wireless charging mechanisms that interact with wireless charging mechanisms beneath the outer surface of a wearable ring device so that the wearable ring device may be charged using energy from the phone battery while the user is holding their phone, and while the user continues to wear the wearable device. In general, charging pads or other charging mechanisms may be placed on (or built into) objects that the user interacts with on a daily basis, such as a phone, a phone case, a computer mouse, a steering wheel of a vehicle, a water bottle, a coffee mug, a glove, another wearable ring device, a watch or other wrist-worn wearable device, and the like.

Being able to charge wearable devices regularly, even while being worn, may enable more consistent data collection. Further, battery/energy constraints of wearable devices may be alleviated with more frequent charging opportunities. In some cases, the wearable device may identify charging patterns enabled by aspects of the present disclosure, and may adjust data collection and/or processing functionality based on the charging patterns. For example, a system associated with a wearable ring device may identify that the ring is likely to be charged at approximately 7:30 am for 30 minutes (as the user holds their steering wheel, with an embedded charging mechanism, while driving to work) and at approximately 5:30 pm for 30 minutes (as the user holds their steering wheel driving home) every weekday. In this example, the wearable ring device may perform physiological measurements with increased power (to improve data quality) due to the probability of an upcoming charging opportunity identified based on the charging pattern.

Aspects of the disclosure are initially described in the context of systems supporting physiological data collection from users via wearable devices. Aspects of the disclosure are then described in the context of a timing diagram. Aspects of the disclosure are further illustrated by and described with reference to apparatus diagrams, system diagrams, and flowcharts that relate to wearable ring devices for on-the-go charging.

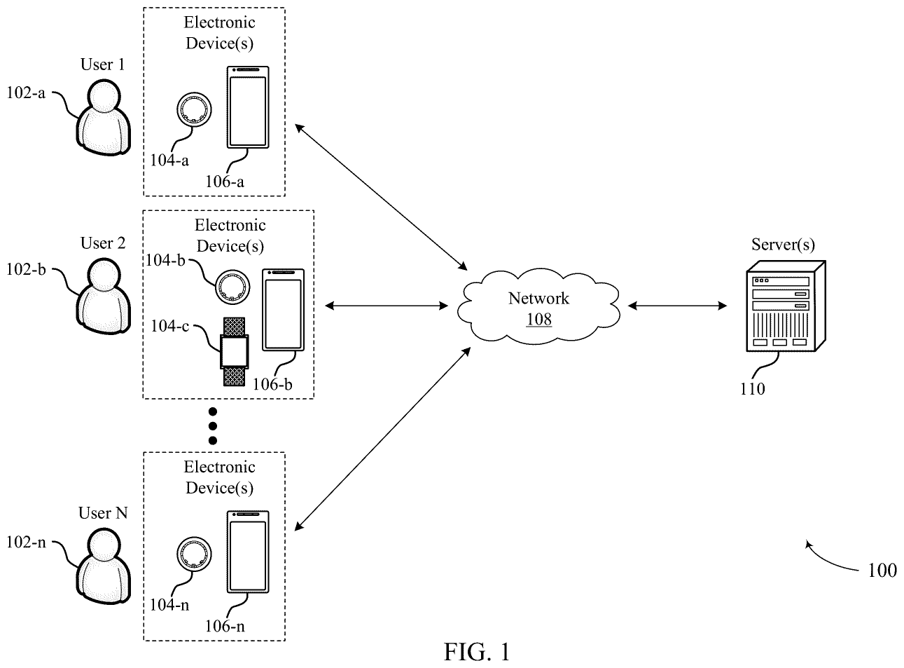

FIG. 1 illustrates an example of a system 100 that supports a wearable ring device for on-the-go charging in accordance with aspects of the present disclosure. The system 100 includes a plurality of electronic devices (e.g., wearable devices 104, user devices 106) that may be worn and/or operated by one or more users 102. The system 100 further includes a network 108 and one or more servers 110.

The electronic devices may include any electronic devices known in the art, including wearable devices 104 (e.g., ring wearable devices, watch wearable devices, etc.), user devices 106 (e.g., smartphones, laptops, tablets). The electronic devices associated with the respective users 102 may include one or more of the following functionalities: 1) measuring physiological data, 2) storing the measured data, 3) processing the data, 4) providing outputs (e.g., via GUIs) to a user 102 based on the processed data, and 5) communicating data with one another and/or other computing devices. Different electronic devices may perform one or more of the functionalities.

Example wearable devices 104 may include wearable computing devices, such as a ring computing device (hereinafter “ring”) configured to be worn on a user's 102 finger, a wrist computing device (e.g., a smart watch, fitness band, or bracelet) configured to be worn on a user's 102 wrist, and/or a head mounted computing device (e.g., glasses/goggles). Wearable devices 104 may also include bands, straps (e.g., flexible or inflexible bands or straps), stick-on sensors, and the like, that may be positioned in other locations, such as bands around the head (e.g., a forehead headband), arm (e.g., a forearm band and/or bicep band), and/or leg (e.g., a thigh or calf band), behind the ear, under the armpit, and the like. Wearable devices 104 may also be attached to, or included in, articles of clothing. For example, wearable devices 104 may be included in pockets and/or pouches on clothing. As another example, wearable device 104 may be clipped and/or pinned to clothing, or may otherwise be maintained within the vicinity of the user 102. Example articles of clothing may include, but are not limited to, hats, shirts, gloves, pants, socks, outerwear (e.g., jackets), and undergarments. In some implementations, wearable devices 104 may be included with other types of devices such as training/sporting devices that are used during physical activity. For example, wearable devices 104 may be attached to, or included in, a bicycle, skis, a tennis racket, a golf club, and/or training weights.

Much of the present disclosure may be described in the context of a ring wearable device 104. Accordingly, the terms “ring 104,” “wearable device 104,” and like terms, may be used interchangeably, unless noted otherwise herein. However, the use of the term “ring 104” is not to be regarded as limiting, as it is contemplated herein that aspects of the present disclosure may be performed using other wearable devices (e.g., watch wearable devices, necklace wearable device, bracelet wearable devices, earring wearable devices, anklet wearable devices, and the like).

In some aspects, user devices 106 may include handheld mobile computing devices, such as smartphones and tablet computing devices. User devices 106 may also include personal computers, such as laptop and desktop computing devices. Other example user devices 106 may include server computing devices that may communicate with other electronic devices (e.g., via the Internet). In some implementations, computing devices may include medical devices, such as external wearable computing devices (e.g., Holter monitors). Medical devices may also include implantable medical devices, such as pacemakers and cardioverter defibrillators. Other example user devices 106 may include home computing devices, such as internet of things (IoT) devices (e.g., IoT devices), smart televisions, smart speakers, smart displays (e.g., video call displays), hubs (e.g., wireless communication hubs), security systems, smart appliances (e.g., thermostats and refrigerators), and fitness equipment.

Some electronic devices (e.g., wearable devices 104, user devices 106) may measure physiological parameters of respective users 102, such as photoplethysmography waveforms, continuous skin temperature, a pulse waveform, respiration rate, heart rate, heart rate variability (HRV), actigraphy, galvanic skin response, pulse oximetry, blood oxygen saturation (SpO2), blood sugar levels (e.g., glucose metrics), and/or other physiological parameters. Some electronic devices that measure physiological parameters may also perform some/all of the calculations described herein. Some electronic devices may not measure physiological parameters, but may perform some/all of the calculations described herein. For example, a ring (e.g., wearable device 104), mobile device application, or a server computing device may process received physiological data that was measured by other devices.

In some implementations, a user 102 may operate, or may be associated with, multiple electronic devices, some of which may measure physiological parameters and some of which may process the measured physiological parameters. In some implementations, a user 102 may have a ring (e.g., wearable device 104) that measures physiological parameters. The user 102 may also have, or be associated with, a user device 106 (e.g., mobile device, smartphone), where the wearable device 104 and the user device 106 are communicatively coupled to one another. In some cases, the user device 106 may receive data from the wearable device 104 and perform some/all of the calculations described herein. In some implementations, the user device 106 may also measure physiological parameters described herein, such as motion/activity parameters.

For example, as illustrated in FIG. 1, a first user 102-a (User 1) may operate, or may be associated with, a wearable device 104-a (e.g., ring 104-a) and a user device 106-a that may operate as described herein. In this example, the user device 106-a associated with user 102-a may process/store physiological parameters measured by the ring 104-a. Comparatively, a second user 102-b (User 2) may be associated with a ring 104-b, a watch wearable device 104-c (e.g., watch 104-c), and a user device 106-b, where the user device 106-b associated with user 102-b may process/store physiological parameters measured by the ring 104-b and/or the watch 104-c. Moreover, an nth user 102-n (User N) may be associated with an arrangement of electronic devices described herein (e.g., ring 104-n, user device 106-n). In some aspects, wearable devices 104 (e.g., rings 104, watches 104) and other electronic devices may be communicatively coupled to the user devices 106 of the respective users 102 via Bluetooth, Wi-Fi, and other wireless protocols.

In some implementations, the rings 104 (e.g., wearable devices 104) of the system 100 may be configured to collect physiological data from the respective users 102 based on arterial blood flow within the user's finger. In particular, a ring 104 may utilize one or more light-emitting components, such as LEDs (e.g., red LEDs, green LEDs) that emit light on the palm-side of a user's finger to collect physiological data based on arterial blood flow within the user's finger. In general, the terms light-emitting components, light-emitting elements, and like terms, may include, but are not limited to, LEDs, micro LEDs, mini LEDs, laser diodes (LDs) (e.g., vertical cavity surface-emitting lasers (VCSELs), and the like.

In some cases, the system 100 may be configured to collect physiological data from the respective users 102 based on blood flow diffused into a microvascular bed of skin with capillaries and arterioles. For example, the system 100 may collect PPG data based on a measured amount of blood diffused into the microvascular system of capillaries and arterioles. In some implementations, the ring 104 may acquire the physiological data using a combination of both green and red LEDs. The physiological data may include any physiological data known in the art including, but not limited to, temperature data, accelerometer data (e.g., movement/motion data), heart rate data, HRV data, blood oxygen level data, or any combination thereof.

The use of both green and red LEDs may provide several advantages over other solutions, as red and green LEDs have been found to have their own distinct advantages when acquiring physiological data under different conditions (e.g., light/dark, active/inactive) and via different parts of the body, and the like. For example, green LEDs have been found to exhibit better performance during exercise. Moreover, using multiple LEDs (e.g., green and red LEDs) distributed around the ring 104 has been found to exhibit superior performance as compared to wearable devices that utilize LEDs that are positioned close to one another, such as within a watch wearable device. Furthermore, the blood vessels in the finger (e.g., arteries, capillaries) are more accessible via LEDs as compared to blood vessels in the wrist. In particular, arteries in the wrist are positioned on the bottom of the wrist (e.g., palm-side of the wrist), meaning only capillaries are accessible on the top of the wrist (e.g., back of hand side of the wrist), where watch wearable devices and similar devices are typically worn. As such, utilizing LEDs and other sensors within a ring 104 has been found to exhibit superior performance as compared to wearable devices worn on the wrist, as the ring 104 may have greater access to arteries (as compared to capillaries), thereby resulting in stronger signals and more valuable physiological data.

The electronic devices of the system 100 (e.g., user devices 106, wearable devices 104) may be communicatively coupled to one or more servers 110 via wired or wireless communication protocols. For example, as shown in FIG. 1, the electronic devices (e.g., user devices 106) may be communicatively coupled to one or more servers 110 via a network 108. The network 108 may implement transfer control protocol and internet protocol (TCP/IP), such as the Internet, or may implement other network 108 protocols. Network connections between the network 108 and the respective electronic devices may facilitate transport of data via email, web, text messages, mail, or any other appropriate form of interaction within a computer network 108. For example, in some implementations, the ring 104-a associated with the first user 102-a may be communicatively coupled to the user device 106-a, where the user device 106-a is communicatively coupled to the servers 110 via the network 108. In additional or alternative cases, wearable devices 104 (e.g., rings 104, watches 104) may be directly communicatively coupled to the network 108.

The system 100 may offer an on-demand database service between the user devices 106 and the one or more servers 110. In some cases, the servers 110 may receive data from the user devices 106 via the network 108, and may store and analyze the data. Similarly, the servers 110 may provide data to the user devices 106 via the network 108. In some cases, the servers 110 may be located at one or more data centers. The servers 110 may be used for data storage, management, and processing. In some implementations, the servers 110 may provide a web-based interface to the user device 106 via web browsers.

In some aspects, the system 100 may detect periods of time that a user 102 is asleep, and classify periods of time that the user 102 is asleep into one or more sleep stages (e.g., sleep stage classification). For example, as shown in FIG. 1, User 102-a may be associated with a wearable device 104-a (e.g., ring 104-a) and a user device 106-a. In this example, the ring 104-a may collect physiological data associated with the user 102-a, including temperature, heart rate, HRV, respiratory rate, and the like. In some aspects, data collected by the ring 104-a may be input to a machine learning classifier, where the machine learning classifier is configured to determine periods of time that the user 102-a is (or was) asleep. Moreover, the machine learning classifier may be configured to classify periods of time into different sleep stages, including an awake sleep stage, a rapid eye movement (REM) sleep stage, a light sleep stage (non-REM (NREM)), and a deep sleep stage (NREM). In some aspects, the classified sleep stages may be displayed to the user 102-a via a GUI of the user device 106-a. Sleep stage classification may be used to provide feedback to a user 102-a regarding the user's sleeping patterns, such as recommended bedtimes, recommended wake-up times, and the like. Moreover, in some implementations, sleep stage classification techniques described herein may be used to calculate scores for the respective user, such as Sleep Scores, Readiness Scores, and the like.

In some aspects, the system 100 may utilize circadian rhythm-derived features to further improve physiological data collection, data processing procedures, and other techniques described herein. The term circadian rhythm may refer to a natural, internal process that regulates an individual's sleep-wake cycle, that repeats approximately every 24 hours. In this regard, techniques described herein may utilize circadian rhythm adjustment models to improve physiological data collection, analysis, and data processing. For example, a circadian rhythm adjustment model may be input into a machine learning classifier along with physiological data collected from the user 102-a via the wearable device 104-a. In this example, the circadian rhythm adjustment model may be configured to “weight,” or adjust, physiological data collected throughout a user's natural, approximately 24-hour circadian rhythm. In some implementations, the system may initially start with a “baseline” circadian rhythm adjustment model, and may modify the baseline model using physiological data collected from each user 102 to generate tailored, individualized circadian rhythm adjustment models that are specific to each respective user 102.

In some aspects, the system 100 may utilize other biological rhythms to further improve physiological data collection, analysis, and processing by phase of these other rhythms. For example, if a weekly rhythm is detected within an individual's baseline data, then the model may be configured to adjust “weights” of data by day of the week. Biological rhythms that may require adjustment to the model by this method include: 1) ultradian (faster than a day rhythms, including sleep cycles in a sleep state, and oscillations from less than an hour to several hours periodicity in the measured physiological variables during wake state; 2) circadian rhythms; 3) non-endogenous daily rhythms shown to be imposed on top of circadian rhythms, as in work schedules; 4) weekly rhythms, or other artificial time periodicities exogenously imposed (e.g. in a hypothetical culture with 12 day “weeks,” 12 day rhythms could be used); 5) multi-day ovarian rhythms in women and spermatogenesis rhythms in men; 6) lunar rhythms (relevant for individuals living with low or no artificial lights); and 7) seasonal rhythms.

The biological rhythms are not always stationary rhythms. For example, many women experience variability in ovarian cycle length across cycles, and ultradian rhythms are not expected to occur at exactly the same time or periodicity across days even within a user. As such, signal processing techniques sufficient to quantify the frequency composition while preserving temporal resolution of these rhythms in physiological data may be used to improve detection of these rhythms, to assign phase of each rhythm to each moment in time measured, and to thereby modify adjustment models and comparisons of time intervals. The biological rhythm-adjustment models and parameters can be added in linear or non-linear combinations as appropriate to more accurately capture the dynamic physiological baselines of an individual or group of individuals.

In some aspects, the respective devices of the system 100 may support “on-the-go” charging of a wearable device 104, such as a ring 104, while the ring 104 is being worn by a user 102. In particular, the ring 104 may support charging mechanisms (e.g., electrical contacts and/or inductive charging components) disposed within an outer surface of the ring 104 such that the ring 104 may charge when positioned next to charging elements positioned on or within everyday objects. For example, cases worn on a user device 106 may be built with wireless charging mechanisms that interact with wireless charging mechanisms beneath the outer surface of a ring 104 (and/or beneath the inner surface of the ring 104) so that the ring 104 may be charged using energy from a battery of the user device 106 while the user 102 is holding the user device 106, and while the user 102 continues to wear the ring 104. In general, charging pads or other charging mechanisms may be placed on (or built into) objects that the user 102 interacts with on a daily basis, such as a user device 106 (e.g., a phone), a case for the user device 106 (e.g., a phone case), a computer mouse, a steering wheel of a vehicle, a water bottle, a coffee mug, a wearable ring device 104, a watch or other wrist-worn wearable device 104, and the like.

Additionally, enabling the ring 104 to be charged while being worn by the user 102 may enable the ring 104 to perform physiological measurements with increased power (e.g., to improve data quality) due to an increased frequency of charging occasions. For example, the ring 104, the user device 106 associated with the ring 104, one or more servers 110 associated with the user device 106, or any combination thereof, may identify charging patterns enabled by the “on-the-go” charging capabilities and may adjust data collection and/or processing functionality based on the charging patterns. For instance, a device of the system 100 may identify that a user 102 with a wearable ring device 104 interacts with a mouse including an embedded charging mechanism during weekdays between 9:00 am and 5:00 μm. The respective device may identify that the wearable ring device 104 of the user experiences charging occasions at an intermittent frequency such that the wearable ring device 104 worn by the user 102 typically achieves a full charge by 5:00 pm. As such, the wearable ring device 104 may perform physiological measurements with increased power prior to and during the 9:00 am to 5:00 pm charging interval due to the probability of intermittent charging opportunities based on the identified charging pattern.

It should be appreciated by a person skilled in the art that one or more aspects of the disclosure may be implemented in a system 100 to additionally or alternatively solve other problems than those described above. Furthermore, aspects of the disclosure may provide technical improvements to “conventional” systems or processes as described herein. However, the description and appended drawings only include example technical improvements resulting from implementing aspects of the disclosure, and accordingly do not represent all of the technical improvements provided within the scope of the claims.

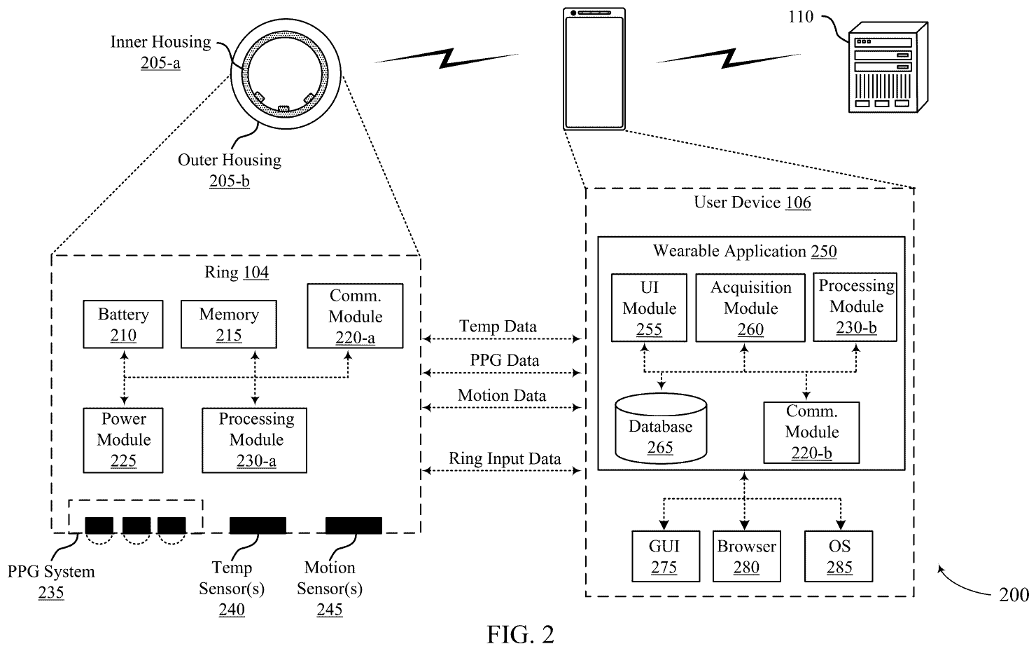

FIG. 2 illustrates an example of a system 200 that supports a wearable ring device for on-the-go charging in accordance with aspects of the present disclosure. The system 200 may implement, or be implemented by, system 100. In particular, system 200 illustrates an example of a ring 104 (e.g., wearable device 104), a user device 106, and a server 110, as described with reference to FIG. 1.

In some aspects, the ring 104 may be configured to be worn around a user's finger, and may determine one or more user physiological parameters when worn around the user's finger. Example measurements and determinations may include, but are not limited to, user skin temperature, pulse waveforms, respiratory rate, heart rate, HRV, blood oxygen levels (SpO2), blood sugar levels (e.g., glucose metrics), and the like.

The system 200 further includes a user device 106 (e.g., a smartphone) in communication with the ring 104. For example, the ring 104 may be in wireless and/or wired communication with the user device 106. In some implementations, the ring 104 may send measured and processed data (e.g., temperature data, photoplethysmogram (PPG) data, motion/accelerometer data, ring input data, and the like) to the user device 106. The user device 106 may also send data to the ring 104, such as ring 104 firmware/configuration updates. The user device 106 may process data. In some implementations, the user device 106 may transmit data to the server 110 for processing and/or storage.

The ring 104 may include a housing 205 that may include an inner housing 205-a and an outer housing 205-b. In some aspects, the housing 205 of the ring 104 may store or otherwise include various components of the ring including, but not limited to, device electronics, a power source (e.g., battery 210, and/or capacitor), one or more substrates (e.g., printable circuit boards) that interconnect the device electronics and/or power source, and the like. The device electronics may include device modules (e.g., hardware/software), such as: a processing module 230-a, a memory 215, a communication module 220-a, a power module 225, and the like. The device electronics may also include one or more sensors. Example sensors may include one or more temperature sensors 240, a PPG sensor assembly (e.g., PPG system 235), and one or more motion sensors 245.

The sensors may include associated modules (not illustrated) configured to communicate with the respective components/modules of the ring 104, and generate signals associated with the respective sensors. In some aspects, each of the components/modules of the ring 104 may be communicatively coupled to one another via wired or wireless connections. Moreover, the ring 104 may include additional and/or alternative sensors or other components that are configured to collect physiological data from the user, including light sensors (e.g., LEDs), oximeters, and the like.

The ring 104 shown and described with reference to FIG. 2 is provided solely for illustrative purposes. As such, the ring 104 may include additional or alternative components as those illustrated in FIG. 2. Other rings 104 that provide functionality described herein may be fabricated. For example, rings 104 with fewer components (e.g., sensors) may be fabricated. In a specific example, a ring 104 with a single temperature sensor 240 (or other sensor), a power source, and device electronics configured to read the single temperature sensor 240 (or other sensor) may be fabricated. In another specific example, a temperature sensor 240 (or other sensor) may be attached to a user's finger (e.g., using adhesives, wraps, clamps, spring loaded clamps, etc.). In this case, the sensor may be wired to another computing device, such as a wrist worn computing device that reads the temperature sensor 240 (or other sensor). In other examples, a ring 104 that includes additional sensors and processing functionality may be fabricated.

The housing 205 may include one or more housing 205 components. The housing 205 may include an outer housing 205-b component (e.g., a shell) and an inner housing 205-a component (e.g., a molding). The housing 205 may include additional components (e.g., additional layers) not explicitly illustrated in FIG. 2. For example, in some implementations, the ring 104 may include one or more insulating layers that electrically insulate the device electronics and other conductive materials (e.g., electrical traces) from the outer housing 205-b (e.g., a metal outer housing 205-b). The housing 205 may provide structural support for the device electronics, battery 210, substrate(s), and other components. For example, the housing 205 may protect the device electronics, battery 210, and substrate(s) from mechanical forces, such as pressure and impacts. The housing 205 may also protect the device electronics, battery 210, and substrate(s) from water and/or other chemicals.

The outer housing 205-b may be fabricated from one or more materials. In some implementations, the outer housing 205-b may include a metal, such as titanium, that may provide strength and abrasion resistance at a relatively light weight. The outer housing 205-b may also be fabricated from other materials, such polymers. In some implementations, the outer housing 205-b may be protective as well as decorative.

The inner housing 205-a may be configured to interface with the user's finger. The inner housing 205-a may be formed from a polymer (e.g., a medical grade polymer) or other material. In some implementations, the inner housing 205-a may be transparent. For example, the inner housing 205-a may be transparent to light emitted by the PPG light emitting diodes (LEDs). In some implementations, the inner housing 205-a component may be molded onto the outer housing 205-b. For example, the inner housing 205-a may include a polymer that is molded (e.g., injection molded) to fit into an outer housing 205-b metallic shell.

The ring 104 may include one or more substrates (not illustrated). The device electronics and battery 210 may be included on the one or more substrates. For example, the device electronics and battery 210 may be mounted on one or more substrates. Example substrates may include one or more printed circuit boards (PCBs), such as flexible PCB (e.g., polyimide). In some implementations, the electronics/battery 210 may include surface mounted devices (e.g., surface-mount technology (SMT) devices) on a flexible PCB. In some implementations, the one or more substrates (e.g., one or more flexible PCBs) may include electrical traces that provide electrical communication between device electronics. The electrical traces may also connect the battery 210 to the device electronics.

The device electronics, battery 210, and substrates may be arranged in the ring 104 in a variety of ways. In some implementations, one substrate that includes device electronics may be mounted along the bottom of the ring 104 (e.g., the bottom half), such that the sensors (e.g., PPG system 235, temperature sensors 240, motion sensors 245, and other sensors) interface with the underside of the user's finger. In these implementations, the battery 210 may be included along the top portion of the ring 104 (e.g., on another substrate).

The various components/modules of the ring 104 represent functionality (e.g., circuits and other components) that may be included in the ring 104. Modules may include any discrete and/or integrated electronic circuit components that implement analog and/or digital circuits capable of producing the functions attributed to the modules herein. For example, the modules may include analog circuits (e.g., amplification circuits, filtering circuits, analog/digital conversion circuits, and/or other signal conditioning circuits). The modules may also include digital circuits (e.g., combinational or sequential logic circuits, memory circuits etc.).

The memory 215 (memory module) of the ring 104 may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device. The memory 215 may store any of the data described herein. For example, the memory 215 may be configured to store data (e.g., motion data, temperature data, PPG data) collected by the respective sensors and PPG system 235. Furthermore, memory 215 may include instructions that, when executed by one or more processing circuits, cause the modules to perform various functions attributed to the modules herein. The device electronics of the ring 104 described herein are only example device electronics. As such, the types of electronic components used to implement the device electronics may vary based on design considerations.

The functions attributed to the modules of the ring 104 described herein may be embodied as one or more processors, hardware, firmware, software, or any combination thereof. Depiction of different features as modules is intended to highlight different functional aspects and does not necessarily imply that such modules must be realized by separate hardware/software components. Rather, functionality associated with one or more modules may be performed by separate hardware/software components or integrated within common hardware/software components.

The processing module 230-a of the ring 104 may include one or more processors (e.g., processing units), microcontrollers, digital signal processors, systems on a chip (SOCs), and/or other processing devices. The processing module 230-a communicates with the modules included in the ring 104. For example, the processing module 230-a may transmit/receive data to/from the modules and other components of the ring 104, such as the sensors. As described herein, the modules may be implemented by various circuit components. Accordingly, the modules may also be referred to as circuits (e.g., a communication circuit and power circuit).

The processing module 230-a may communicate with the memory 215. The memory 215 may include computer-readable instructions that, when executed by the processing module 230-a, cause the processing module 230-a to perform the various functions attributed to the processing module 230-a herein. In some implementations, the processing module 230-a (e.g., a microcontroller) may include additional features associated with other modules, such as communication functionality provided by the communication module 220-a (e.g., an integrated Bluetooth Low Energy transceiver) and/or additional onboard memory 215.

The communication module 220-a may include circuits that provide wireless and/or wired communication with the user device 106 (e.g., communication module 220-b of the user device 106). In some implementations, the communication modules 220-a, 220-b may include wireless communication circuits, such as Bluetooth circuits and/or Wi-Fi circuits. In some implementations, the communication modules 220-a, 220-b can include wired communication circuits, such as Universal Serial Bus (USB) communication circuits. Using the communication module 220-a, the ring 104 and the user device 106 may be configured to communicate with each other. The processing module 230-a of the ring may be configured to transmit/receive data to/from the user device 106 via the communication module 220-a. Example data may include, but is not limited to, motion data, temperature data, pulse waveforms, heart rate data, HRV data, PPG data, and status updates (e.g., charging status, battery charge level, and/or ring 104 configuration settings). The processing module 230-a of the ring may also be configured to receive updates (e.g., software/firmware updates) and data from the user device 106.

The ring 104 may include a battery 210 (e.g., a rechargeable battery 210). An example battery 210 may include a Lithium-Ion or Lithium-Polymer type battery 210, although a variety of battery 210 options are possible. The battery 210 may be wirelessly charged. In some implementations, the ring 104 may include a power source other than the battery 210, such as a capacitor. The power source (e.g., battery 210 or capacitor) may have a curved geometry that matches the curve of the ring 104. In some aspects, a charger or other power source may include additional sensors that may be used to collect data in addition to, or that supplements, data collected by the ring 104 itself. Moreover, a charger or other power source for the ring 104 may function as a user device 106, in which case the charger or other power source for the ring 104 may be configured to receive data from the ring 104, store and/or process data received from the ring 104, and communicate data between the ring 104 and the servers 110.

In some aspects, the ring 104 includes a power module 225 that may control charging of the battery 210. For example, the power module 225 may interface with an external wireless charger that charges the battery 210 when interfaced with the ring 104. The charger may include a datum structure that mates with a ring 104 datum structure to create a specified orientation with the ring 104 during charging. The power module 225 may also regulate voltage(s) of the device electronics, regulate power output to the device electronics, and monitor the state of charge of the battery 210. In some implementations, the battery 210 may include a protection circuit module (PCM) that protects the battery 210 from high current discharge, over voltage during charging, and under voltage during discharge. The power module 225 may also include electro-static discharge (ESD) protection.

The one or more temperature sensors 240 may be electrically coupled to the processing module 230-a. The temperature sensor 240 may be configured to generate a temperature signal (e.g., temperature data) that indicates a temperature read or sensed by the temperature sensor 240. The processing module 230-a may determine a temperature of the user in the location of the temperature sensor 240. For example, in the ring 104, temperature data generated by the temperature sensor 240 may indicate a temperature of a user at the user's finger (e.g., skin temperature). In some implementations, the temperature sensor 240 may contact the user's skin. In other implementations, a portion of the housing 205 (e.g., the inner housing 205-a) may form a barrier (e.g., a thin, thermally conductive barrier) between the temperature sensor 240 and the user's skin. In some implementations, portions of the ring 104 configured to contact the user's finger may have thermally conductive portions and thermally insulative portions. The thermally conductive portions may conduct heat from the user's finger to the temperature sensors 240. The thermally insulative portions may insulate portions of the ring 104 (e.g., the temperature sensor 240) from ambient temperature.

In some implementations, the temperature sensor 240 may generate a digital signal (e.g., temperature data) that the processing module 230-a may use to determine the temperature. As another example, in cases where the temperature sensor 240 includes a passive sensor, the processing module 230-a (or a temperature sensor 240 module) may measure a current/voltage generated by the temperature sensor 240 and determine the temperature based on the measured current/voltage. Example temperature sensors 240 may include a thermistor, such as a negative temperature coefficient (NTC) thermistor, or other types of sensors including resistors, transistors, diodes, and/or other electrical/electronic components.

The processing module 230-a may sample the user's temperature over time. For example, the processing module 230-a may sample the user's temperature according to a sampling rate. An example sampling rate may include one sample per second, although the processing module 230-a may be configured to sample the temperature signal at other sampling rates that are higher or lower than one sample per second. In some implementations, the processing module 230-a may sample the user's temperature continuously throughout the day and night. Sampling at a sufficient rate (e.g., one sample per second) throughout the day may provide sufficient temperature data for analysis described herein.

The processing module 230-a may store the sampled temperature data in memory 215. In some implementations, the processing module 230-a may process the sampled temperature data. For example, the processing module 230-a may determine average temperature values over a period of time. In one example, the processing module 230-a may determine an average temperature value each minute by summing all temperature values collected over the minute and dividing by the number of samples over the minute. In a specific example where the temperature is sampled at one sample per second, the average temperature may be a sum of all sampled temperatures for one minute divided by sixty seconds. The memory 215 may store the average temperature values over time. In some implementations, the memory 215 may store average temperatures (e.g., one per minute) instead of sampled temperatures in order to conserve memory 215.

The sampling rate, that may be stored in memory 215, may be configurable. In some implementations, the sampling rate may be the same throughout the day and night. In other implementations, the sampling rate may be changed throughout the day/night. In some implementations, the ring 104 may filter/reject temperature readings, such as large spikes in temperature that are not indicative of physiological changes (e.g., a temperature spike from a hot shower). In some implementations, the ring 104 may filter/reject temperature readings that may not be reliable due to other factors, such as excessive motion during exercise (e.g., as indicated by a motion sensor 245).

The ring 104 (e.g., communication module) may transmit the sampled and/or average temperature data to the user device 106 for storage and/or further processing. The user device 106 may transfer the sampled and/or average temperature data to the server 110 for storage and/or further processing.

Although the ring 104 is illustrated as including a single temperature sensor 240, the ring 104 may include multiple temperature sensors 240 in one or more locations, such as arranged along the inner housing 205-a near the user's finger. In some implementations, the temperature sensors 240 may be stand-alone temperature sensors 240. Additionally, or alternatively, one or more temperature sensors 240 may be included with other components (e.g., packaged with other components), such as with the accelerometer and/or processor.

The processing module 230-a may acquire and process data from multiple temperature sensors 240 in a similar manner described with respect to a single temperature sensor 240. For example, the processing module 230 may individually sample, average, and store temperature data from each of the multiple temperature sensors 240. In other examples, the processing module 230-a may sample the sensors at different rates and average/store different values for the different sensors. In some implementations, the processing module 230-a may be configured to determine a single temperature based on the average of two or more temperatures determined by two or more temperature sensors 240 in different locations on the finger.

The temperature sensors 240 on the ring 104 may acquire distal temperatures at the user's finger (e.g., any finger). For example, one or more temperature sensors 240 on the ring 104 may acquire a user's temperature from the underside of a finger or at a different location on the finger. In some implementations, the ring 104 may continuously acquire distal temperature (e.g., at a sampling rate). Although distal temperature measured by a ring 104 at the finger is described herein, other devices may measure temperature at the same/different locations. In some cases, the distal temperature measured at a user's finger may differ from the temperature measured at a user's wrist or other external body location. Additionally, the distal temperature measured at a user's finger (e.g., a “shell” temperature) may differ from the user's core temperature. As such, the ring 104 may provide a useful temperature signal that may not be acquired at other internal/external locations of the body. In some cases, continuous temperature measurement at the finger may capture temperature fluctuations (e.g., small or large fluctuations) that may not be evident in core temperature. For example, continuous temperature measurement at the finger may capture minute-to-minute or hour-to-hour temperature fluctuations that provide additional insight that may not be provided by other temperature measurements elsewhere in the body.

The ring 104 may include a PPG system 235. The PPG system 235 may include one or more optical transmitters that transmit light. The PPG system 235 may also include one or more optical receivers that receive light transmitted by the one or more optical transmitters. An optical receiver may generate a signal (hereinafter “PPG” signal) that indicates an amount of light received by the optical receiver. The optical transmitters may illuminate a region of the user's finger. The PPG signal generated by the PPG system 235 may indicate the perfusion of blood in the illuminated region. For example, the PPG signal may indicate blood volume changes in the illuminated region caused by a user's pulse pressure. The processing module 230-a may sample the PPG signal and determine a user's pulse waveform based on the PPG signal. The processing module 230-a may determine a variety of physiological parameters based on the user's pulse waveform, such as a user's respiratory rate, heart rate, HRV, oxygen saturation, and other circulatory parameters.

In some implementations, the PPG system 235 may be configured as a reflective PPG system 235 where the optical receiver(s) receive transmitted light that is reflected through the region of the user's finger. In some implementations, the PPG system 235 may be configured as a transmissive PPG system 235 where the optical transmitter(s) and optical receiver(s) are arranged opposite to one another, such that light is transmitted directly through a portion of the user's finger to the optical receiver(s).

The number and ratio of transmitters and receivers included in the PPG system 235 may vary. Example optical transmitters may include light-emitting diodes (LEDs). The optical transmitters may transmit light in the infrared spectrum and/or other spectrums. Example optical receivers may include, but are not limited to, photosensors, phototransistors, and photodiodes. The optical receivers may be configured to generate PPG signals in response to the wavelengths received from the optical transmitters. The location of the transmitters and receivers may vary. Additionally, a single device may include reflective and/or transmissive PPG systems 235.

The PPG system 235 illustrated in FIG. 2 may include a reflective PPG system 235 in some implementations. In these implementations, the PPG system 235 may include a centrally located optical receiver (e.g., at the bottom of the ring 104) and two optical transmitters located on each side of the optical receiver. In this implementation, the PPG system 235 (e.g., optical receiver) may generate the PPG signal based on light received from one or both of the optical transmitters. In other implementations, other placements, combinations, and/or configurations of one or more optical transmitters and/or optical receivers are contemplated.

The processing module 230-a may control one or both of the optical transmitters to transmit light while sampling the PPG signal generated by the optical receiver. In some implementations, the processing module 230-a may cause the optical transmitter with the stronger received signal to transmit light while sampling the PPG signal generated by the optical receiver. For example, the selected optical transmitter may continuously emit light while the PPG signal is sampled at a sampling rate (e.g., 250 Hz).

Sampling the PPG signal generated by the PPG system 235 may result in a pulse waveform that may be referred to as a “PPG.” The pulse waveform may indicate blood pressure vs time for multiple cardiac cycles. The pulse waveform may include peaks that indicate cardiac cycles. Additionally, the pulse waveform may include respiratory induced variations that may be used to determine respiration rate. The processing module 230-a may store the pulse waveform in memory 215 in some implementations. The processing module 230-a may process the pulse waveform as it is generated and/or from memory 215 to determine user physiological parameters described herein.

The processing module 230-a may determine the user's heart rate based on the pulse waveform. For example, the processing module 230-a may determine heart rate (e.g., in beats per minute) based on the time between peaks in the pulse waveform. The time between peaks may be referred to as an interbeat interval (IBI). The processing module 230-a may store the determined heart rate values and IBI values in memory 215.

The processing module 230-a may determine HRV over time. For example, the processing module 230-a may determine HRV based on the variation in the IBIs. The processing module 230-a may store the HRV values over time in the memory 215. Moreover, the processing module 230-a may determine the user's respiratory rate over time. For example, the processing module 230-a may determine respiratory rate based on frequency modulation, amplitude modulation, or baseline modulation of the user's IBI values over a period of time. Respiratory rate may be calculated in breaths per minute or as another breathing rate (e.g., breaths per 30 seconds). The processing module 230-a may store user respiratory rate values over time in the memory 215.

The ring 104 may include one or more motion sensors 245, such as one or more accelerometers (e.g., 6-D accelerometers) and/or one or more gyroscopes (gyros). The motion sensors 245 may generate motion signals that indicate motion of the sensors. For example, the ring 104 may include one or more accelerometers that generate acceleration signals that indicate acceleration of the accelerometers. As another example, the ring 104 may include one or more gyro sensors that generate gyro signals that indicate angular motion (e.g., angular velocity) and/or changes in orientation. The motion sensors 245 may be included in one or more sensor packages. An example accelerometer/gyro sensor is a Bosch BMI160 inertial micro electro-mechanical system (MEMS) sensor that may measure angular rates and accelerations in three perpendicular axes.

The processing module 230-a may sample the motion signals at a sampling rate (e.g., 50 Hz) and determine the motion of the ring 104 based on the sampled motion signals. For example, the processing module 230-a may sample acceleration signals to determine acceleration of the ring 104. As another example, the processing module 230-a may sample a gyro signal to determine angular motion. In some implementations, the processing module 230-a may store motion data in memory 215. Motion data may include sampled motion data as well as motion data that is calculated based on the sampled motion signals (e.g., acceleration and angular values).

The ring 104 may store a variety of data described herein. For example, the ring 104 may store temperature data, such as raw sampled temperature data and calculated temperature data (e.g., average temperatures). As another example, the ring 104 may store PPG signal data, such as pulse waveforms and data calculated based on the pulse waveforms (e.g., heart rate values, IBI values, HRV values, and respiratory rate values). The ring 104 may also store motion data, such as sampled motion data that indicates linear and angular motion.

The ring 104, or other computing device, may calculate and store additional values based on the sampled/calculated physiological data. For example, the processing module 230 may calculate and store various metrics, such as sleep metrics (e.g., a Sleep Score), activity metrics, and readiness metrics. In some implementations, additional values/metrics may be referred to as “derived values.” The ring 104, or other computing/wearable device, may calculate a variety of values/metrics with respect to motion. Example derived values for motion data may include, but are not limited to, motion count values, regularity values, intensity values, metabolic equivalence of task values (METs), and orientation values. Motion counts, regularity values, intensity values, and METs may indicate an amount of user motion (e.g., velocity/acceleration) over time. Orientation values may indicate how the ring 104 is oriented on the user's finger and if the ring 104 is worn on the left hand or right hand.

In some implementations, motion counts and regularity values may be determined by counting a number of acceleration peaks within one or more periods of time (e.g., one or more 30 second to 1 minute periods). Intensity values may indicate a number of movements and the associated intensity (e.g., acceleration values) of the movements. The intensity values may be categorized as low, medium, and high, depending on associated threshold acceleration values. METs may be determined based on the intensity of movements during a period of time (e.g., 30 seconds), the regularity/irregularity of the movements, and the number of movements associated with the different intensities.

In some implementations, the processing module 230-a may compress the data stored in memory 215. For example, the processing module 230-a may delete sampled data after making calculations based on the sampled data. As another example, the processing module 230-a may average data over longer periods of time in order to reduce the number of stored values. In a specific example, if average temperatures for a user over one minute are stored in memory 215, the processing module 230-a may calculate average temperatures over a five minute time period for storage, and then subsequently erase the one minute average temperature data. The processing module 230-a may compress data based on a variety of factors, such as the total amount of used/available memory 215 and/or an elapsed time since the ring 104 last transmitted the data to the user device 106.

Although a user's physiological parameters may be measured by sensors included on a ring 104, other devices may measure a user's physiological parameters. For example, although a user's temperature may be measured by a temperature sensor 240 included in a ring 104, other devices may measure a user's temperature. In some examples, other wearable devices (e.g., wrist devices) may include sensors that measure user physiological parameters. Additionally, medical devices, such as external medical devices (e.g., wearable medical devices) and/or implantable medical devices, may measure a user's physiological parameters. One or more sensors on any type of computing device may be used to implement the techniques described herein.

The physiological measurements may be taken continuously throughout the day and/or night. In some implementations, the physiological measurements may be taken during portions of the day and/or portions of the night. In some implementations, the physiological measurements may be taken in response to determining that the user is in a specific state, such as an active state, resting state, and/or a sleeping state. For example, the ring 104 can make physiological measurements in a resting/sleep state in order to acquire cleaner physiological signals. In one example, the ring 104 or other device/system may detect when a user is resting and/or sleeping and acquire physiological parameters (e.g., temperature) for that detected state. The devices/systems may use the resting/sleep physiological data and/or other data when the user is in other states in order to implement the techniques of the present disclosure.

In some implementations, as described previously herein, the ring 104 may be configured to collect, store, and/or process data, and may transfer any of the data described herein to the user device 106 for storage and/or processing. In some aspects, the user device 106 includes a wearable application 250, an operating system (OS) 285, a web browser application (e.g., web browser 280), one or more additional applications, and a GUI 275. The user device 106 may further include other modules and components, including sensors, audio devices, haptic feedback devices, and the like. The wearable application 250 may include an example of an application (e.g., “app”) that may be installed on the user device 106. The wearable application 250 may be configured to acquire data from the ring 104, store the acquired data, and process the acquired data as described herein. For example, the wearable application 250 may include a user interface (UI) module 255, an acquisition module 260, a processing module 230-b, a communication module 220-b, and a storage module (e.g., database 265) configured to store application data.

The various data processing operations described herein may be performed by the ring 104, the user device 106, the servers 110, or any combination thereof. For example, in some cases, data collected by the ring 104 may be pre-processed and transmitted to the user device 106. In this example, the user device 106 may perform some data processing operations on the received data, may transmit the data to the servers 110 for data processing, or both. For instance, in some cases, the user device 106 may perform processing operations that require relatively low processing power and/or operations that require a relatively low latency, whereas the user device 106 may transmit the data to the servers 110 for processing operations that require relatively high processing power and/or operations that may allow relatively higher latency.

In some aspects, the ring 104, user device 106, and server 110 of the system 200 may be configured to evaluate sleep patterns for a user. In particular, the respective components of the system 200 may be used to collect data from a user via the ring 104, and generate one or more scores (e.g., Sleep Score, Readiness Score) for the user based on the collected data. For example, as noted previously herein, the ring 104 of the system 200 may be worn by a user to collect data from the user, including temperature, heart rate, HRV, and the like. Data collected by the ring 104 may be used to determine when the user is asleep in order to evaluate the user's sleep for a given “sleep day.” In some aspects, scores may be calculated for the user for each respective sleep day, such that a first sleep day is associated with a first set of scores, and a second sleep day is associated with a second set of scores. Scores may be calculated for each respective sleep day based on data collected by the ring 104 during the respective sleep day. Scores may include, but are not limited to, Sleep Scores, Readiness Scores, and the like.

In some cases, “sleep days” may align with the traditional calendar days, such that a given sleep day runs from midnight to midnight of the respective calendar day. In other cases, sleep days may be offset relative to calendar days. For example, sleep day's may run from 6:00 pm (18:00) of a calendar day until 6:00 pm (18:00) of the subsequent calendar day. In this example, 6:00 pm may serve as a “cut-off time,” where data collected from the user before 6:00 pm is counted for the current sleep day, and data collected from the user after 6:00 pm is counted for the subsequent sleep day. Due to the fact that most individuals sleep the most at night, offsetting sleep days relative to calendar days may enable the system 200 to evaluate sleep patterns for users in such a manner that is consistent with their sleep schedules. In some cases, users may be able to selectively adjust (e.g., via the GUI) a timing of sleep days relative to calendar days so that the sleep days are aligned with the duration of time that the respective users typically sleep.

In some implementations, each overall score for a user for each respective day (e.g., Sleep Score, Readiness Score) may be determined/calculated based on one or more “contributors,” “factors,” or “contributing factors.” For example, a user's overall Sleep Score may be calculated based on a set of contributors, including: total sleep, efficiency, restfulness, REM sleep, deep sleep, latency, timing, or any combination thereof. The Sleep Score may include any quantity of contributors. The “total sleep” contributor may refer to the sum of all sleep periods of the sleep day. The “efficiency” contributor may reflect the percentage of time spent asleep compared to time spent awake while in bed, and may be calculated using the efficiency average of long sleep periods (e.g., primary sleep period) of the sleep day, weighted by a duration of each sleep period. The “restfulness” contributor may indicate how restful the user's sleep is, and may be calculated using the average of all sleep periods of the sleep day, weighted by a duration of each period. The restfulness contributor may be based on a “wake up count” (e.g., sum of all the wake-ups (when user wakes up) detected during different sleep periods), excessive movement, and a “got up count” (e.g., sum of all the got-ups (when user gets out of bed) detected during the different sleep periods).

The “REM sleep” contributor may refer to a sum total of REM sleep durations across all sleep periods of the sleep day including REM sleep. Similarly, the “deep sleep” contributor may refer to a sum total of deep sleep durations across all sleep periods of the sleep day including deep sleep. The “latency” contributor may signify how long (e.g., average, median, longest) the user takes to go to sleep, and may be calculated using the average of long sleep periods throughout the sleep day, weighted by a duration of each period and the number of such periods (e.g., consolidation of a given sleep stage or sleep stages may be its own contributor or weight other contributors). Lastly, the “timing” contributor may refer to a relative timing of sleep periods within the sleep day and/or calendar day, and may be calculated using the average of all sleep periods of the sleep day, weighted by a duration of each period.

By way of another example, a user's overall Readiness Score may be calculated based on a set of contributors, including: sleep, sleep balance, heart rate, HRV balance, recovery index, temperature, activity, activity balance, or any combination thereof. The Readiness Score may include any quantity of contributors. The “sleep” contributor may refer to the combined Sleep Score of all sleep periods within the sleep day. The “sleep balance” contributor may refer to a cumulative duration of all sleep periods within the sleep day. In particular, sleep balance may indicate to a user whether the sleep that the user has been getting over some duration of time (e.g., the past two weeks) is in balance with the user's needs. Typically, adults need 7-9 hours of sleep a night to stay healthy, alert, and to perform at their best both mentally and physically. However, it is normal to have an occasional night of bad sleep, so the sleep balance contributor takes into account long-term sleep patterns to determine whether each user's sleep needs are being met. The “resting heart rate” contributor may indicate a lowest heart rate from the longest sleep period of the sleep day (e.g., primary sleep period) and/or the lowest heart rate from naps occurring after the primary sleep period.

Continuing with reference to the “contributors” (e.g., factors, contributing factors) of the Readiness Score, the “HRV balance” contributor may indicate a highest HRV average from the primary sleep period and the naps happening after the primary sleep period. The HRV balance contributor may help users keep track of their recovery status by comparing their HRV trend over a first time period (e.g., two weeks) to an average HRV over some second, longer time period (e.g., three months). The “recovery index” contributor may be calculated based on the longest sleep period. Recovery index measures how long it takes for a user's resting heart rate to stabilize during the night. A sign of a very good recovery is that the user's resting heart rate stabilizes during the first half of the night, at least six hours before the user wakes up, leaving the body time to recover for the next day. The “body temperature” contributor may be calculated based on the longest sleep period (e.g., primary sleep period) or based on a nap happening after the longest sleep period if the user's highest temperature during the nap is at least 0.5° C. higher than the highest temperature during the longest period. In some aspects, the ring may measure a user's body temperature while the user is asleep, and the system 200 may display the user's average temperature relative to the user's baseline temperature. If a user's body temperature is outside of their normal range (e.g., clearly above or below 0.0), the body temperature contributor may be highlighted (e.g., go to a “Pay attention” state) or otherwise generate an alert for the user.

In some aspects, the system 200 may support techniques for “on-the-go” charging of a wearable device 104, such as a ring 104, while the ring 104 is being worn by a user 102. In particular, as described previously, the ring 104 may include the housing 205 that includes the inner housing 205-a (e.g., inner surface) configured to contact a tissue of a user 102 while the ring 104 is being worn by the user 102, and an outer housing 205-b (e.g., outer surface) that is opposite the inner housing 205-a. Additionally, the ring 104 may include a battery 210 and one or more sensors, such as the PPG system 235, the temperature sensors 240, and the motion sensors 245, positioned at least partially within the housing 205. Further, the ring 104 may include a wireless charging component (e.g., coupled with the battery 210) disposed at least partially within the housing 205, where the wireless charging component is configured to perform an inductive charging procedure, a contact-based charging procedure, or both, to transfer energy from an external power source to the battery 210 through the outer housing 205-b and/or inner housing 205-a of the ring 104 while the ring 104 is being worn by the user 102. For example, the wireless charging component, an additional wireless charging component, or both, may transfer energy from a charging device to the battery 210 through the outer housing 205-b and/or inner housing 205-a while the ring 104 is positioned on or within a proximity of the charging device.

For example (e.g., in the case of contact-based charging), the outer housing 205-b of the ring 104 may include one or more contact points (e.g., electrical contact points or surfaces) that, when physically contacting a charging element (e.g., wireless charger, charging pad, or the like thereof) of a charging device, may enable wireless energy transfer to charge the battery 210 of the ring 104. In another example (e.g., in the case of inductive charging), the housing 205 of the ring 104 may include one or more inductive charging elements that, when positioned within a threshold proximity of or contacting a charging element of a charging device, may enable wireless energy transfer to charge the battery 210 of the ring 104.

In general, charging elements (e.g., charging mechanism) may be placed on (e.g., or built into) objects that the user 102 interacts with on a daily basis (e.g., or with increased frequency). For example, a case worn on the user device 106 may be built with wireless charging mechanisms that interact with wireless charging mechanisms of the ring 104 so that the battery 210 of the ring 104 may be charged using energy from a battery of the user device 106 while the user 102 is holding the user device 106, and while the user 102 continues to wear the ring 104. Additional examples of objects that may include charging elements may include a user device 106 (e.g., a phone), a case for the user device 106 (e.g., a phone case), a computer mouse, a steering wheel of a vehicle, the handlebars of a bicycle and/or motorcycle, handles of exercise equipment (e.g., handlebars of a stationary bike, handles of a stair-climber, dumbbells, etc.), a water bottle or coffee mug, a glove, another wearable ring device 104, a watch or other wrist-worn wearable device 104, and the like.

Additionally, enabling the ring 104 to be charged while being worn by the user 102 may enable the ring 104 to perform physiological measurements with increased power (e.g., to improve data quality) due to an increased frequency in charging occasions. For example, one or more processing modules 230, such as the processing module 230-a of the wireless device, the processing module 230-b of the user device 106, or both, may identify charging patterns enabled by the wireless charging capabilities of the ring 104 and may adjust data collection and/or processing functionality based on the charging patterns. In some examples, the processing module 230-b of the user device 106 may identify the charging patterns and may transmit, via the respective communication modules 220, an indication of the charging patterns to the processing module 230-a, the memory 215, or both, of the ring 104.

That is, the one or more processing modules 230 may identify a charging pattern associated with the wireless charging component based on one or more prior energy transfer occasions between a charging device and the battery 210 and may adjust at least one measurement parameter used by the one or more sensors to acquire physiological data based on one or more characteristics of the charging pattern. In such cases, the one or more characteristics of the charging pattern may include a timing of energy transfer occasions between the external power source and the battery, a frequency of energy transfer occasions between the charging device and the battery 210, or both.

For example, a component of the system 100 may identify that, during weekdays between 9:00 am and 5:00 pm, a user 102 interacts with a mouse with an embedded charging mechanism (e.g., a charging device) at an intermittent frequency, such that a ring 104 worn by the user 102 typically achieves a full charge by 5:00 pm. As such, the one or more processing modules may generate a first set of instructions configured to cause the one or more sensors to acquire physiological data from the user 102 according to a first set of parameters associated with increased power consumption between 9:00 am and 5:00 pm (e.g., during a first time interval) based on the probability of intermittent charging opportunities (e.g., wireless energy transfer) between 9:00 am and 5:00 μm. In other words, the respective devices of system 100 may determine that the ring 104 is likely to be charged between 9:00 am and 5:00 pm on weekdays, and may therefore increase a power used to perform measurements and/or perform additional types of measurements due to the increased likelihood of a charging occasion during such times. Additionally, or alternatively, the one or more processing modules may generate a second set of instructions configured to cause the one or more sensors to acquire physiological data from the user 102 according to a second set of parameters associated with lower power consumption (e.g., relative to the first set of parameters) outside of 9:00 am and 5:00 pm (e.g., during a second time interval).

In some examples, adjusting the set of parameters from the first set of parameters to the second set of parameters (e.g., or vise-versa) may include increasing a current or voltage applied to a light-emitting component, selectively activating at least one sensor of the one or more sensors, selectively adjusting a type of measurement performed by the one or more sensors, or any combination thereof.

Though the “on-the-go” charging techniques of the present disclosure are primarily described in the context of an inductive charging procedure, a contact-based charging procedure, or both, this is not to be regarded as a limitation of the present disclosure. In this regard, any charging procedure that may enable wireless charging between a wearable device, such as the ring 104, and a charging device while the ring 104 is worn by a user 102 may be implemented according to aspects of the present disclosure, such as charging via transmission of energy signaling (e.g., radio frequency charging).

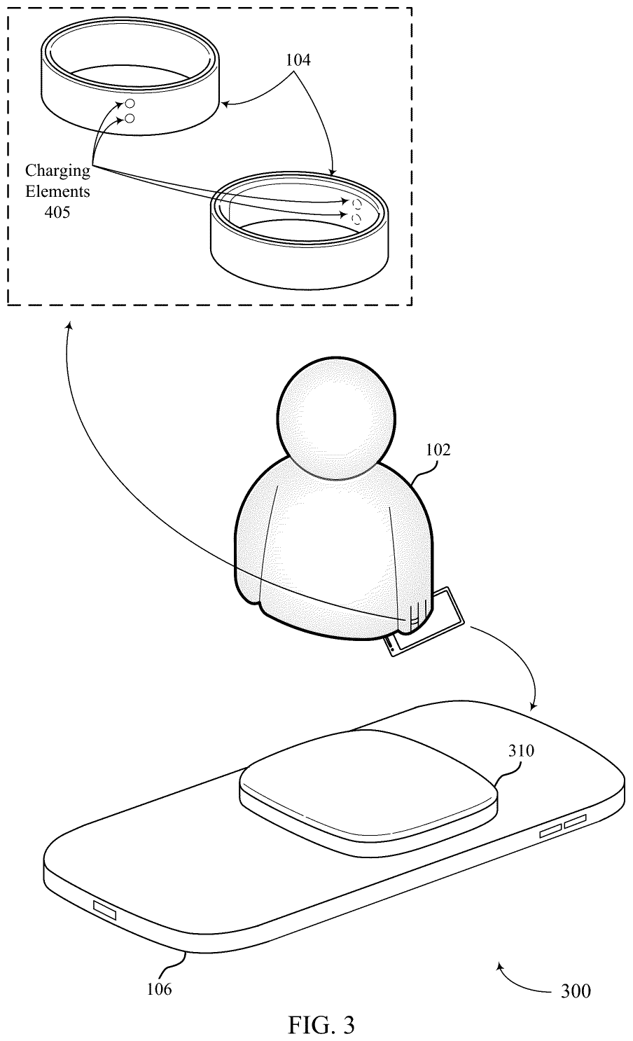

FIG. 3 shows an example of a system 300 that supports a wearable ring device for on-the-go charging in accordance with aspects of the present disclosure. The system 300 may implement, or be implemented by, system 100, system 200, or both. In particular, system 300 illustrates an example of a ring 104 (e.g., wearable device 104) and a user device, as described with reference to FIG. 1.

In some aspects, the system 300 may support techniques for “on-the-go” charging of a wearable device 104, such as a ring 104, while the ring 104 is being worn by a user 102. In particular, the ring 104 may include a housing, made up of an inner surface and an outer surface opposite the inner surface, where the housing includes one or more electrical components. For example, a battery and one or more sensors (e.g., electrically coupled to the battery) may be positioned at least partially within the housing, where the one or more sensors are configured to collect physiological data from the user 102.

Additionally, the one or more electrical components positioned at least partially within the housing may include one or more charging elements 405 of a wireless charging component. The one or more charging elements 405 may enable the wireless charging component to perform a charging procedure to wirelessly transfer energy from an external power source, such as a wireless charger 310, to the battery while the ring 104 is worn by the user 102. In other words, a hand of the user 102 wearing the ring 104 may be positioned near (e.g., be in contact with) one or more external power sources intermittently throughout a day, such that the ring 104 may receive energy, or receive a charge, from the one or more external power sources via the one or more charging elements 405 of the wireless charging component. In some examples, the entire outer surface may be considered a charging element 405.

As will be described in further detail herein, the charging elements 405 may include contact-based charging elements, inductive-based charging elements, or both. Moreover, as shown in FIG. 3, the wearable device 104 may include charging elements 405 positioned on an outer surface of the wearable device 104, the inner surface of the wearable device 104, or both. In such cases, charging elements 405 positioned on the outer and/or inner surfaces may facilitate charging procedures for the wearable device 104 through the outer surface and/or the inner surface, respectively. In this regard, charging elements 405 positioned on the outer surface of the wearable device 104 may facilitate charging procedures through the outer surface while the wearable device 104 is being worn. Comparatively, charging elements 405 positioned on the inner surface of the wearable device 104 may facilitate charging procedures through the inner surface while the wearable device 104 is placed on a charger, or otherwise not being worn.

As described previously, the one or more external power sources may include a wireless charger 310 (e.g., a wireless charging component) that may be located on or within (e.g., built into) everyday objects that the user 102 may interact with. For example, as depicted in FIG. 3, a case on a user device 106 held by the user 102 may include a wireless charger 310, such that the charging elements 405 may receive energy from the wireless charger 310 when the user 102 holds the user device 106.

In general, wireless charger 310 (e.g., charging pad or other charging mechanisms) may be placed on objects that the user 102 interacts with on a daily basis, such as the user device 106, the case of the user device 106, a computer mouse, a key board, a steering wheel of a vehicle, a steering wheel cover, a water bottle or coffee mug, a glove, another wearable ring device 104, a watch or other wrist-worn wearable device 104, and the like. In some examples, the wireless charger 310 may transfer energy to the ring 104 based on a power level of the wireless charger 310 (and/or power level of the associated user device 106) satisfying a threshold. That is, the wireless charger 310 may refrain from transferring energy to the 104 based on the power level of the wireless charger 310 and/or user device 106 being below the threshold (e.g., charging for the wearable device 104 is halted when the wireless charger 310 and/or user device 106 does not have sufficient power).

In some examples, the charging procedure supported by the wireless charging component including the charging elements 405 may be a contact-based charging procedure. In such cases, the one or more charging elements 405 may receive energy from the wireless charger 310 based on the charging elements 405 contacting one or more contact-based charging elements 405 of the wireless charger 310. Additionally, or alternatively, the charging procedure supported by the wireless charging component including the charging elements 405 may be an inductive charging procedure. In such cases, the one or more charging elements 405 may receive energy from the wireless charger 310 based on the one or more charging elements being located within a threshold proximity of one or more inductive charging elements 405 of the wireless charger 310.

In additional or alternative implementations, the wireless charger 310 may be configured to charge the wearable device 104 via a photovoltaic charging procedure. For example, in some implementations, the wearable device 104 may include one or more photovoltaic cells, and the charger device 310 may include one or more light-emitting components (e.g., lasers, LEDs, etc.) that are configured to direct light to the photovoltaic cells to perform photovoltaic charging. In some cases, PPG sensors (e.g., photodiodes) of the wearable device 104 may also be used or repurposed for photovoltaic charging.

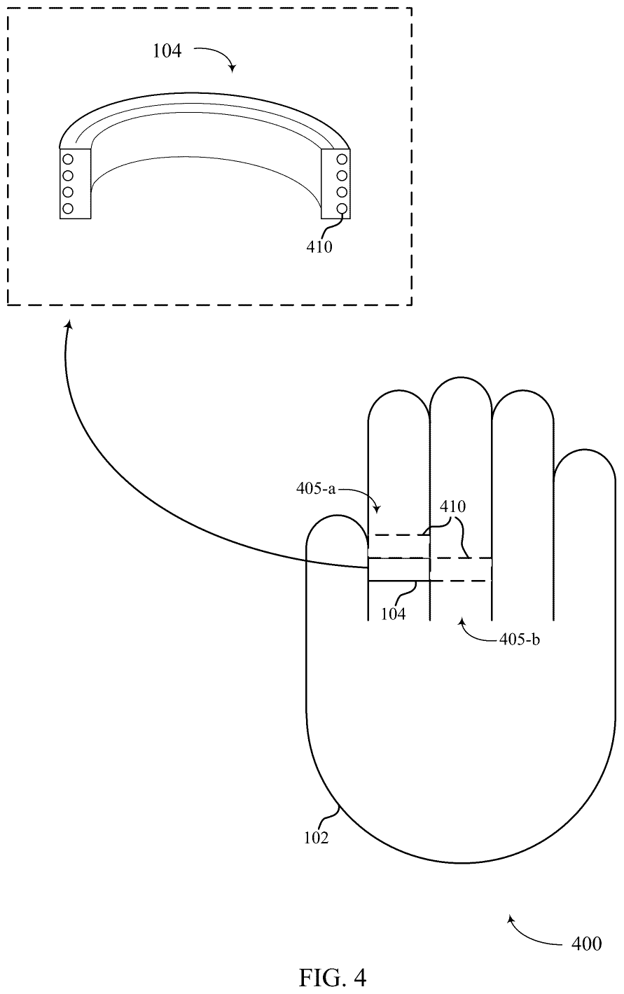

FIG. 4 shows an example of a system 400 that supports a wearable ring device for on-the-go charging in accordance with aspects of the present disclosure. The system 400 may implement, or be implemented by, system 100, system 200, the system 300, or any combination thereof. In particular, system 400 illustrates an example of a ring 104 (e.g., wearable device 104), as described with reference to FIG. 1.

As shown in FIG. 4, the system 400 may include a wearable ring device 104 (e.g., data collection ring 104) that is configured to acquire physiological data from a user 102, and a charging ring 410 that is configured to facilitate charging of the wearable ring device 104. In this regard, the wearable ring device 104 may support charging mechanisms (e.g., electrical contacts and/or inductive charging components) that enable the wearable ring device 104 to charge when positioned next to charging elements positioned on or within the charging ring 410. For example, the charging ring 410 may include one or more charging mechanisms, such that the wearable ring device 104 may be charging using energy from a battery of the charging ring 410. That is, the charging ring 410 may be worn at a position 405-a or a position 405-b, such that the charging ring 410 wirelessly transfers energy to the wearable ring device 104 while the wearable ring device 104 is worn by the user and continues to collect physiological data from the user 102.

In some examples, the charging ring 410 may wirelessly transfer energy to the wearable ring device 104 via an induction charging procedure. As such, the position 405-a and the position 405-b may be located within a proximity of the wearable ring device 104 to enable the inductive charging procedure. For instance, the charging ring 410 may be positioned on the same finger as the wearable ring device (position 405-a) and/or an adjacent finger (position 405-b) to enable a wireless charging procedure between the charging ring 410 and the wearable ring device 104. In some cases, the wearable ring device 104 and/or charging ring 410 may include magnetic components configured to attract to one another in order to position the rings close enough to one another to facilitate the induction-based charging procedure.

In some other examples, the charging ring 410 may wirelessly transfer energy to the wearable ring device 104 via a contact-based charging procedure. In such cases, the wearable ring device 104 and the charging ring 410 may include one or more magnetic elements, such that a first set of magnetic elements on the wearable ring device 104 magnetically connect with a second set of magnetic elements on the charging ring 410 to help position the respective rings in a correct orientation relative to one another to facilitate the contact-based charging procedure.

In this regard, the one or more magnetic elements of the wearable ring device 104 and the charging ring 410 may help align the rings in a manner that supports efficient wireless charging. That is, each ring may include one or more charging coils 410 associated with the respective wireless charging mechanisms. In such cases, alignment of a first set of charging coils 410 (e.g., inductive receiving coils) within the wearable ring device 104 with a second set of charging coils 410 (e.g., inductive transmitting coils) in the charging ring 410 may support efficient energy transfer through coupling of the sets of charging coils 410. Each set of coils 410 may be positioned within the respective housing and may wrap around the respective ring, as depicted in the cross-sectional view in FIG. 4.

Additionally, or alternatively, one or more physical features of each ring may support alignment (e.g., and contact) of the rings. That is, one or more physical features on the wearable ring device 104 may interlock or otherwise engage with one or more physical features on the charging ring 410 to help align/orient the rings relative to one another in an orientation that facilitates wireless charging. In such cases, the interlocking may enable alignment of the first set of charging coils 410 in the wearable ring 104 with the second set of charging coils 410 in the charging ring 410 to facilitate wireless charging procedures.

In some examples, the charging ring 410 may be a second wearable ring device 104. That is, each wearable ring device 104 may support charging mechanisms that are capable of both receiving charge (e.g., energy) from another wearable ring device 104, as well as transferring charge to another wearable ring device 104. Stated differently, the “charging” ring could also include new sensor elements that extend the measurement capabilities of the ring system.

For example, a first user 102 may be associated with a first wearable ring device 104 and a second user 102 may be associated with a second wearable device 104. In some cases, the first wearable ring device 104 may have low battery and the second user 102 may offer the second wearable ring device 104 to the first user 102 to use to charge the first wearable ring device 102. As such, the first user 102 may place the second wearable ring device 104 at the position 405-a or the position 405-b to enable the second wearable ring device 104 to wirelessly transfer energy to the first wearable ring device 104. Similarly, the second wearable ring device 104 may have low battery and the first user 102 may offer the first wearable ring device 104 to the second user 102 to use to charge the second wearable ring device 102. As such, the second user 102 may place the first wearable ring device 104 at the position 405-a or the position 405-b to enable the first wearable ring device 104 to wirelessly transfer energy to the second wearable ring device 104. In either scenario, the first user 102 or the second user 102 may input, into a user device 106 associated with the first wearable ring device 104 or the second wearable ring device 104, respectively, an indication that the respective wearable ring device 104 is being worn by another user 102, such that the respective wearable ring device 104 may refrain from acquiring physiological data from a different user 102.

By way of another example, blood oxygen saturation measurements (e.g., SpO2 measurements) may be performed by a second wearable device 104 that is configured to charge the first wearable device 104. For instance, if the first wearable device 104 is manufactured to be smaller and thinner for aesthetic and/or comfortability purposes, the first wearable device 104 may not exhibit the power capacity, battery capacity, and/or components (e.g., red/IR LEDs) that are needed for accurate SpO2 measurement. As such, this SpO2 functionality may be moved to the second/charging wearable device 104. Further, because SpO2 monitoring may not be used daily, sufficient SpO2 monitoring may still be performed by occasionally pairing the first wearable device 104 with a second/charging wearable device 104 with SpO2 monitoring functionality. Moreover, in some cases, the components used for SpO2 monitoring may be split across the first/second wearable devices 104. For instance, the first wearable device 104 may include photodetectors for PPG measurements, such that the second/charging wearable device 104 does not need or include photodetectors for SpO2 monitoring. In such cases, the second/charging wearable device 104 may include red/IR LEDs, and the first wearable device 104 may include photodetectors, such that SpO2 measurements are performed by transmitting light from the second/charging wearable device 104 and receiving the light via the photodetectors of the first wearable device 104. In this regard, the respective wearable devices 104 may be synchronized and used as a single system for SpO2 measurement.

Additionally, or alternatively, the wearable ring devices 104 may be configured to automatically perform a charging procedure where the ring with the higher battery level transfers energy to the ring with the lower battery level. Further, in some cases, the the wearable ring devices 104 may be configured to perform the charging procedure until such point that the ring being charged rises above some threshold battery level, and/or until the ring performing the charging (e.g., the ring transferring energy to the other ring) falls below some additional threshold battery level.

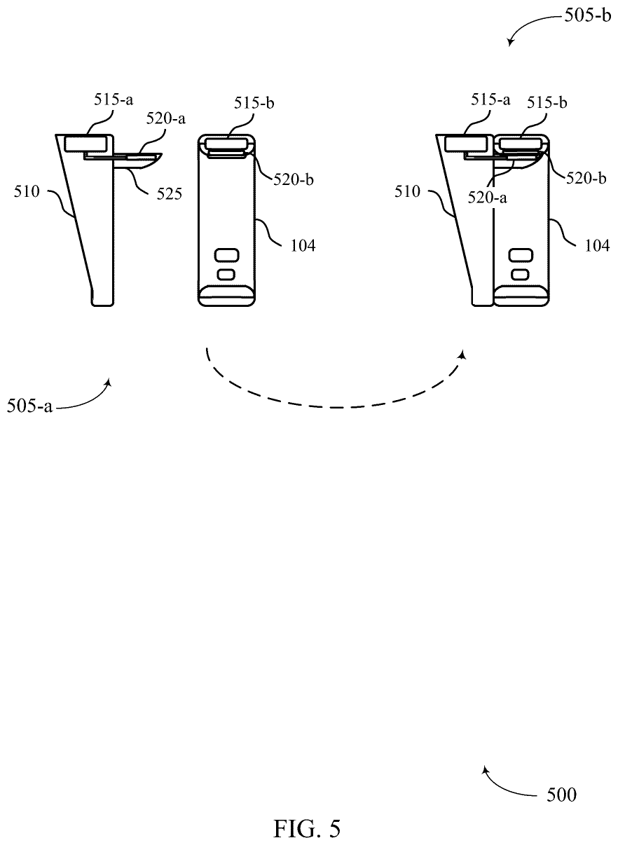

FIG. 5 shows an example of a system 500 that supports a wearable ring device for on-the-go charging in accordance with aspects of the present disclosure. The system 500 may implement, or be implemented by, system 100, system 200, the system 300, the system 400, or any combination thereof. In particular, system 500 illustrates an example of a ring 104 (e.g., wearable device 104), as described with reference to FIG. 1.

As described previously with reference to FIG. 4, in some examples, a ring 104 may receive wireless energy transferred from a charging ring 510 (e.g., a charging ring 410). That is, as shown in FIG. 5, the system 500 may include a ring 104 (e.g., data collection ring 104) that is configured to acquire physiological data from a user 102, and a charging ring 510 that is configured to facilitate charging of the wearable ring device 104. In this regard, the ring 104 may support charging mechanisms (e.g., electrical contacts and/or inductive charging components) that enable the ring 104 to charge when positioned next to charging elements positioned on or within the charging ring 510.

For example, the charging ring 510 may include one or more charging mechanisms, such that the ring 104 (e.g., a battery 515-b of the ring 104) may be charged using energy from a battery 515-a of the charging ring 510. That is, as depicted in view 505-a, the charging ring 510 may be positioned on a finger of a user 102, such that a wing 525 aligns with an inner circumference of the ring 104. Thus, as depicted in view 505-b, a user 102 may further position the charging ring 510 on the finger of the user 102 such that the wing 525 is positioned within an inner circumference of the ring 104 (e.g., may push the wing 525 under the ring 104). As such, the charging ring 510 may wirelessly transfer energy to the ring 104 while the ring 104 is worn by the user 102 and continues to collect physiological data from the user 102 (e.g., such as during power-hungry applications, such as during collection of an exercise heart rate or other more power-hungry measurements). In some examples, inclusion of the wing 525 within the inner circumference of the ring 104 may cause the ring 104 to fit tighter on the finger of the user 102 (e.g., as compared to without the wing 525).

In some examples, the charging ring 510 may wirelessly transfer energy to the wearable ring device 104 via an inductive charging procedure. That is, positioning the wing 525 within the inner circumference of the ring 104 may enable a set of induction coils 520-a (e.g., a set of charging coils) of the charging ring 510 to align with a set of induction coils 520-b of the ring 104. In such cases, alignment of a set of induction coils 520-b (e.g., inductive receiving coils) within the ring 104 with the set of induction coils 520-a (e.g., inductive transmitting coils) in the charging ring 510 may support efficient energy transfer through coupling of the sets of induction coils 520. The set of induction coils 520-b may be positioned within the housing of the ring 104, such that the set of induction coils 530-b may wrap around the ring 104. In some examples, one or more magnetic elements of the ring 104 and the charging ring 510 may enable the charging ring 510 (e.g., the wing 525) to maintain position relative to the ring 104.

Additionally, or alternatively, the charging ring 510 may include a display interface (not shown), such as a GUI. The display interface may enable the charging ring 510 to display information received from the ring 104. That is, the ring 104 may communicate with the charging ring 510 via the sets of induction coils 520 (e.g., sets of data transfer coils) and/or via other wireless communication protocols (e.g., Bluetooth), such that data may be transferred from the ring 104 to the charging ring 510. For example, the ring 104 may transfer data associated with a heart rate of the user 102 or an SpO2 value of the user 102 to the charging ring 510, such that the charging ring 510 may display an indication of the heart rate or the SpO2 value to the user 102. Such display capabilities may enable the user 102 to view data associated with the user 102 without use of a user device 106 (e.g., when handling the user device 105 may be difficult, such as during running). In general, the display interface of the charging ring 510 may be configured to display the same or different information as compared to the GUI 275 of the user device 106 configured to execute the wearable application 250.

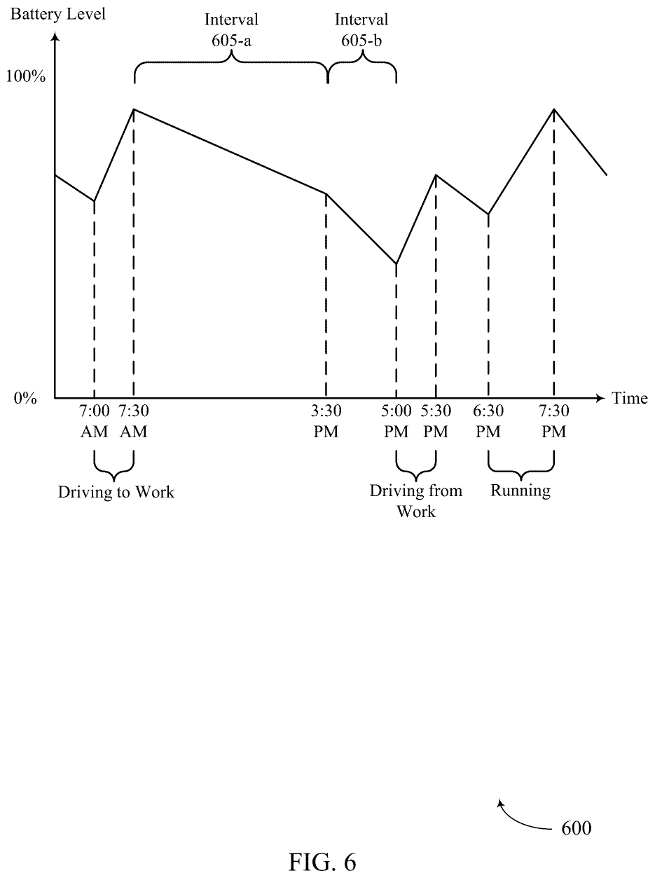

FIG. 6 shows an example of a timing diagram 600 that supports a wearable ring device for on-the-go charging in accordance with aspects of the present disclosure. The timing diagram 600 may implement, or be implemented by, system 100, system 200, the system 300, the system 400, the system 500, or any combination thereof. In particular, timing diagram 600 illustrates an example of one or more charging patterns associated with a ring 104 (e.g., wearable device 104), as described with reference to FIG. 1.

Wireless charging of a wearable device, such as a ring 104, while being worn by a user 102 may alleviate battery/energy constraints and may enable the ring 104 to adjust parameters associated with data collection based on one or more expected charging opportunities. In other words, the ring 104 (e.g., or a system associated with the ring 104) may identify a charging pattern associated with the ring 104 and/or user 102 and may adjust data collection and/or processing functionality based on the charging pattern. In such cases, the ring 104 may consider one or more characteristics of the charging pattern when adjusting the data collection and/or the processing functionality. The one or more characteristics may include a timing of energy transfer occasions between the external power source and the battery of the ring 104, a frequency of energy transfer occasions between the external power source and the battery of the ring 104, or both.

For example, a steering wheel (e.g., or steering wheel cover) of a vehicle driven by a user 102 may include a wireless charging mechanism that may wirelessly transfer energy to a ring 104 while the ring 104 is in contact with (or otherwise close to) the steering wheel. In this example, as shown in the timing diagram 600, the ring 104 (e.g., or a system associated with the ring 104) may identify that, on weekdays, the ring 104 typically receives a charge between 7:00 am and 7:30 am, and then again between 5:00 μm and 5:30 pm. That is, a routine of the user 102 may include driving to work each weekday morning between 7:00 am and 7:30 am, and driving home from work each evening between 5:00 μm and 5:30 pm.

As such, the ring 104 (e.g., or a system associated with the ring 104) may adjust one or more measurement parameters associated with data collection by the ring 104 based on expected charging opportunities between 7:00 am and 7:30 am and between 5:00 μm and 5:30 pm (e.g., based on the charging pattern). In other words, the ring 104 may generate a first set of instructions configured to cause one or more sensors on the ring 104 to collect (e.g., acquire) physiological data from the user 102 during an interval 605-a according to a first set of measurement parameters. Additionally, the ring 104 may identify an upcoming charging opportunity, such as between 5:00 μm and 5:30 μm, and may generate a second set of instructions configured to cause the one or more sensors on the ring 104 to collect physiological data from the user 102 during an interval 605-b according to a second set of measurement parameters. In such cases, the second set of measurement parameters may be associated with an increased power consumption (e.g., and increased accuracy of the collected physiological data) due to the expected upcoming charging opportunity. In some examples, the ring 104 may generate the second set of instructions based on being within a threshold duration of the expected charging opportunity. Additionally, or alternatively, adjusting the first set of measurement parameters to the second set of measurement parameters may include increasing a current or voltage applied to a light-emitting component, selectively activating at least one sensor of the one or more sensors, selectively adjusting a type of measurement performed by the one or more sensors, or any combination thereof.

In another example, the user 102 may routinely run in the evening between 6:30 μm to 7:30 μm and may hold a user device 106 with an embedded charging mechanism during the run. As such, the ring 104 may adjust one or more measurement parameters associated with data collection during the run from one or more first measurement parameters associated with low power consumption to one or more second measurement parameters associated with higher power consumption, such that the ring 104 may collect data at an increased accuracy (e.g., as compared to data collection associated with the one or more first measurement parameters). In other words, the ring 104 may increase power consumption of the ring 104 during the run to support data collection at an increased accuracy based on the ring 104 receiving energy throughout the duration of the run.

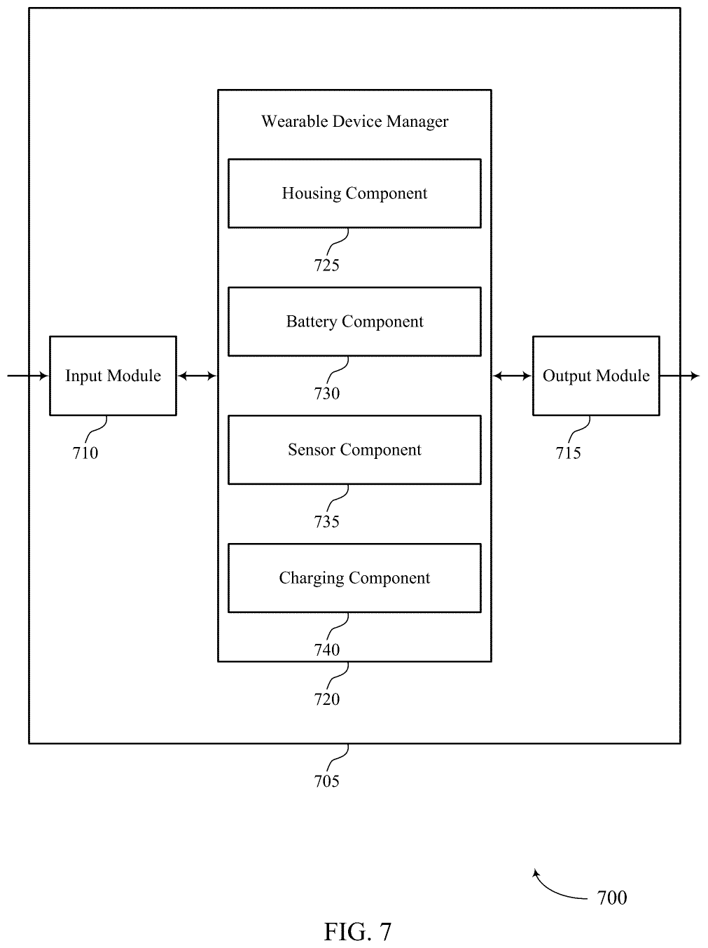

FIG. 7 shows a block diagram 700 of a device 705 that supports a wearable ring device for on-the-go charging in accordance with aspects of the present disclosure. The device 705 may include an input module 710, an output module 715, and a wearable device manager 720. The device 705, or one or more components of the device 705 (e.g., the input module 710, the output module 715, and the wearable device manager 720), may also include at least one processor, that may be coupled with at least one memory, to support the described techniques. Each of these components may be in communication with one another (e.g., via one or more buses).

For example, the wearable device manager 720 may include a housing component 725, a battery component 730, a sensor component 735, a charging component 740, or any combination thereof. In some examples, the wearable device manager 720, or various components thereof, may be configured to perform various operations (e.g., receiving, monitoring, transmitting) using or otherwise in cooperation with the input module 710, the output module 715, or both. For example, the wearable device manager 720 may receive information from the input module 710, send information to the output module 715, or be integrated in combination with the input module 710, the output module 715, or both to receive information, transmit information, or perform various other operations as described herein.

The housing component 725 may be configured as or otherwise support a means for a housing comprising an inner surface and an outer surface that is opposite the inner surface, wherein the inner surface is configured to contact a tissue of a user while the wearable device is being worn by the user. The battery component 730 may be configured as or otherwise support a means for a battery positioned at least partially within the housing. The sensor component 735 may be configured as or otherwise support a means for one or more sensors positioned at least partially within the housing and electrically coupled with the battery, the one or more sensors configured to acquire physiological data from the user through one or more apertures in the inner surface while the wearable device is being worn by the user. The charging component 740 may be configured as or otherwise support a means for a wireless charging component disposed at least partially within the housing, wherein the wireless charging component is configured to perform an inductive charging procedure, a contact-based charging procedure, or both, to wirelessly transfer energy from an external power source to the battery through the outer surface of the wearable device while the wearable device is being worn by the user.

Additionally, or alternatively, the wearable device manager 720 may support charging a wearable device in accordance with examples as disclosed herein. The sensor component 735 may be configured as or otherwise support a means for transmitting light through one or more apertures of an inner surface of the wearable device via one or more light-emitting components of the wearable device, wherein the light is transmitted during a first time interval that the wearable device is being worn by a user. The sensor component 735 may be configured as or otherwise support a means for receiving the light through the one or more apertures via one or more photodetectors during the first time interval. The sensor component 735 may be configured as or otherwise support a means for generating physiological data associated with the user based at least in part on the light received by the one or more photodetectors during the first time interval. The charging component 740 may be configured as or otherwise support a means for wirelessly transferring energy from an external power source to a battery of the wearable device through an outer surface of the wearable device during the first time interval that the wearable device is being worn by the user, wherein the energy is wirelessly transferred using an inductive charging procedure, a contact-based charging procedure, or both.

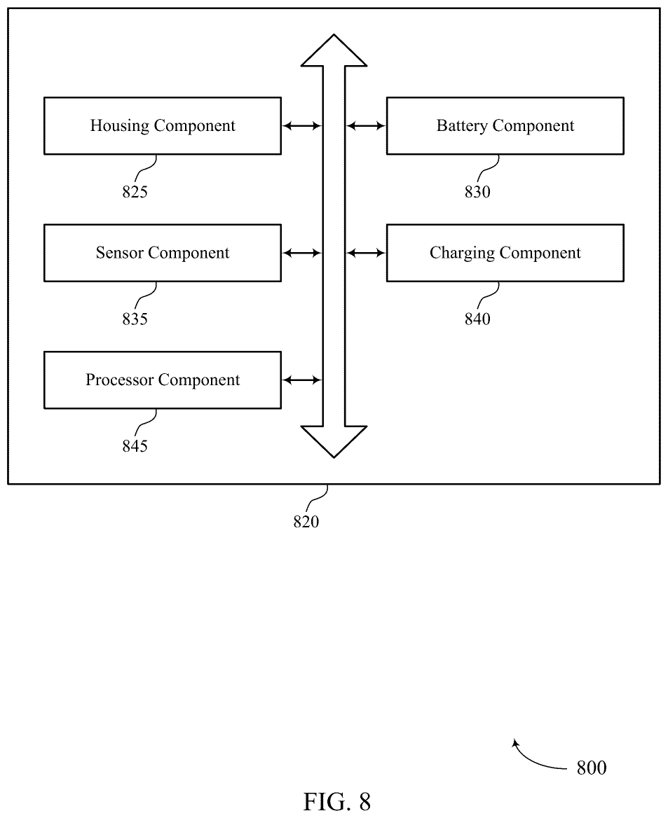

FIG. 8 shows a block diagram 800 of a wearable device manager 820 that supports a wearable ring device for on-the-go charging in accordance with aspects of the present disclosure. The wearable device manager 820 may be an example of aspects of a wearable device manager or a wearable device manager 720, or both, as described herein. The wearable device manager 820, or various components thereof, may be an example of means for performing various aspects of wearable ring device for on-the-go charging as described herein. For example, the wearable device manager 820 may include a housing component 825, a battery component 830, a sensor component 835, a charging component 840, a processor component 845, or any combination thereof. Each of these components, or components of subcomponents thereof (e.g., one or more processors, one or more memories), may communicate, directly or indirectly, with one another (e.g., via one or more buses).

The housing component 825 may be configured as or otherwise support a means for a housing comprising an inner surface and an outer surface that is opposite the inner surface, wherein the inner surface is configured to contact a tissue of a user while the wearable device is being worn by the user. The battery component 830 may be configured as or otherwise support a means for a battery positioned at least partially within the housing. The sensor component 835 may be configured as or otherwise support a means for one or more sensors positioned at least partially within the housing and electrically coupled with the battery, the one or more sensors configured to acquire physiological data from the user through one or more apertures in the inner surface while the wearable device is being worn by the user. The charging component 840 may be configured as or otherwise support a means for a wireless charging component disposed at least partially within the housing, wherein the wireless charging component is configured to perform an inductive charging procedure, a contact-based charging procedure, or both, to wirelessly transfer energy from an external power source to the battery through the outer surface of the wearable device while the wearable device is being worn by the user.

In some examples, the wireless charging component, an additional wireless charging component, or both, is further configured to wirelessly transfer additional energy from a charging device to the battery through the inner surface of the wearable device while the wearable device is positioned on or within the charging device.

In some examples, the processor component 845 may be configured as or otherwise support a means for generating a first set of instructions configured to cause the one or more sensors to acquire the physiological data from the user during a first time interval. In some examples, the processor component 845 may be configured as or otherwise support a means for generating a second set of instructions configured to cause the wireless charging component to wirelessly transfer the energy from the external power source to the battery during the first time interval.

In some examples, selectively adjust at least one measurement parameter used by the one or more sensors to acquire the physiological data based at least in part on the wireless charging component being configured to wireless transfer the energy during the first time interval that the physiological data is acquired.

In some examples, selectively adjusting the at least one measurement parameter comprises increasing a current or voltage applied to a light-emitting component, selectively activating at least one sensor of the one or more sensors, selectively adjusting a type of measurement performed by the one or more sensors, or any combination thereof.

In some examples, the processor component 845 may be configured as or otherwise support a means for determine a charging pattern associated with the wireless charging component based at least in part on one or more prior energy transfer occasions between the external power source and the battery. In some examples, the processor component 845 may be configured as or otherwise support a means for selectively adjust at least one measurement parameter used by the one or more sensors to acquire the physiological data based at least in part on one or more characteristics of the charging pattern.

In some examples, the one or more characteristics of the charging pattern comprise a timing of energy transfer occasions between the external power source and the battery, a frequency of energy transfer occasions between the external power source and the battery, or both.

In some examples, the wireless charging component comprises a first inductive charging component disposed beneath or at least partially within the outer surface of the housing. In some examples, the first inductive charging component is configured to wirelessly interface with a second inductive charging component of the external power source to wirelessly transfer the energy.

In some examples, the wireless charging component comprises a first electrical contact component within the outer surface of the housing. In some examples, the first electrical contact component is configured to physically contact a second electrical contact component of the external power source to wirelessly transfer the energy.

In some examples, the wireless charging component is configured to wirelessly transfer the energy from the external power source to the battery based at least in part on a power level of the external power source satisfying a threshold power level.

In some examples, the external power source is disposed on or within a mobile phone, a case associated with the mobile phone, a steering wheel, a steering wheel cover, a coffee mug, a water bottle, a computer mouse, a keyboard, a glove, a watch wearable device, or a wearable ring device.

In some examples, the wearable device comprises a wearable ring device. In some examples, the inner surface and the outer surface of the housing comprise an inner circumferential surface and an outer circumferential surface, respectively.

In some examples, the wireless charging component is further configured to wirelessly transfer energy from the external power source to the battery through the outer surface of the wearable device while the wearable device is not being worn by the user.

In some examples, one or more coils positioned within the housing and coupled with the wireless charging component, the one or more coils are configured to electrically couple with one or more second coils associated with the external power source to support wireless transfer of energy.

In some examples, the one or more coils wrap around an inner circumferential surface of the housing.

Additionally, or alternatively, the wearable device manager 820 may support charging a wearable device in accordance with examples as disclosed herein. In some examples, the sensor component 835 may be configured as or otherwise support a means for transmitting light through one or more apertures of an inner surface of the wearable device via one or more light-emitting components of the wearable device, wherein the light is transmitted during a first time interval that the wearable device is being worn by a user. In some examples, the sensor component 835 may be configured as or otherwise support a means for receiving the light through the one or more apertures via one or more photodetectors during the first time interval. In some examples, the sensor component 835 may be configured as or otherwise support a means for generating physiological data associated with the user based at least in part on the light received by the one or more photodetectors during the first time interval. In some examples, the charging component 840 may be configured as or otherwise support a means for wirelessly transferring energy from an external power source to a battery of the wearable device through an outer surface of the wearable device during the first time interval that the wearable device is being worn by the user, wherein the energy is wirelessly transferred using an inductive charging procedure, a contact-based charging procedure, or both.

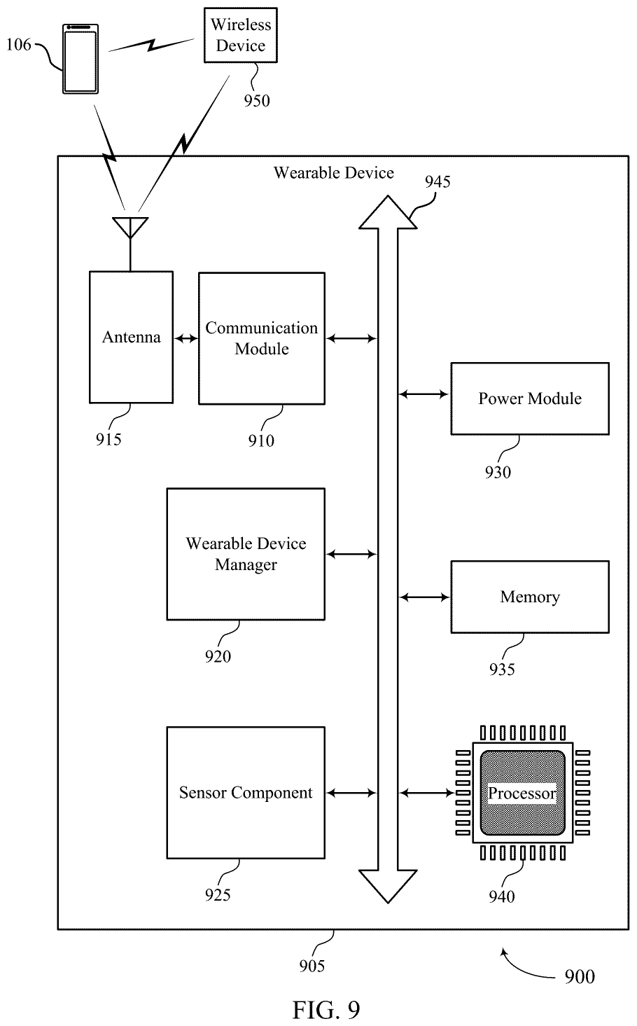

FIG. 9 shows a diagram of a system 900 including a device 905 that supports a wearable ring device for on-the-go charging in accordance with aspects of the present disclosure. The device 905 may be an example of or include the components of a device 705 as described herein. The device 905 may include an example of a wearable device 104, as described previously herein. The device 905 may include components for bi-directional communications including components for transmitting and receiving communications with a user device 106 and a server 110, such as a wearable device manager 920, a communication module 910, an antenna 915, a sensor component 925, a power module 930, a memory 935, a processor 940, and a wireless device 950. These components may be in electronic communication or otherwise coupled (e.g., operatively, communicatively, functionally, electronically, electrically) via one or more buses (e.g., a bus 945).

For example, the wearable device manager 920 may be configured as or otherwise support a means for a housing comprising an inner surface and an outer surface that is opposite the inner surface, wherein the inner surface is configured to contact a tissue of a user while the wearable device is being worn by the user. The wearable device manager 920 may be configured as or otherwise support a means for a battery positioning at least partially within the housing. The wearable device manager 920 may be configured as or otherwise support a means for one or more sensors positioning at least partially within the housing and electrically coupled with the battery, the one or more sensors configured to acquire physiological data from the user through one or more apertures in the inner surface while the wearable device is being worn by the user. The wearable device manager 920 may be configured as or otherwise support a means for a wireless charging component disposed at least partially within the housing, wherein the wireless charging component is configured to perform an inductive charging procedure, a contact-based charging procedure, or both, to wirelessly transfer energy from an external power source to the battery through the outer surface of the wearable device while the wearable device is being worn by the user.

Additionally, or alternatively, the wearable device manager 920 may support charging a wearable device in accordance with examples as disclosed herein. For example, the wearable device manager 920 may be configured as or otherwise support a means for transmitting light through one or more apertures of an inner surface of the wearable device via one or more light-emitting components of the wearable device, wherein the light is transmitted during a first time interval that the wearable device is being worn by a user. The wearable device manager 920 may be configured as or otherwise support a means for receiving the light through the one or more apertures via one or more photodetectors during the first time interval. The wearable device manager 920 may be configured as or otherwise support a means for generating physiological data associated with the user based at least in part on the light received by the one or more photodetectors during the first time interval. The wearable device manager 920 may be configured as or otherwise support a means for wirelessly transferring energy from an external power source to a battery of the wearable device through an outer surface of the wearable device during the first time interval that the wearable device is being worn by the user, wherein the energy is wirelessly transferred using an inductive charging procedure, a contact-based charging procedure, or both.

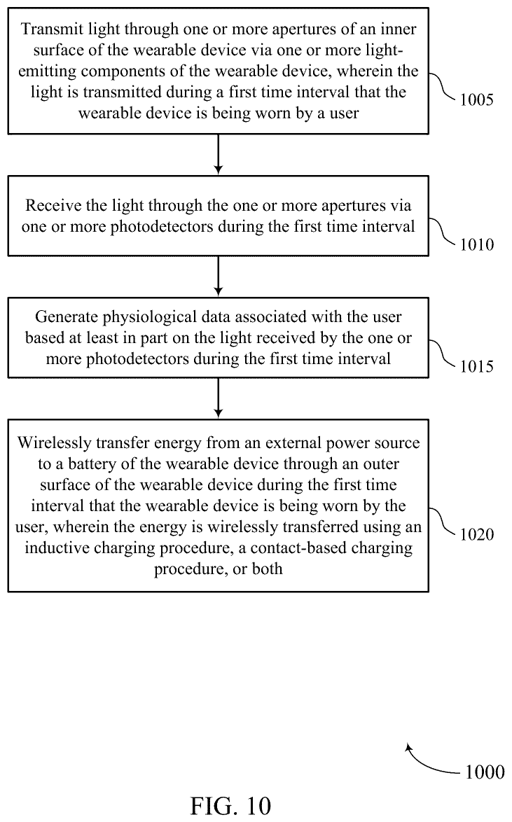

FIG. 10 shows a flowchart illustrating a method 1000 that supports a wearable ring device for on-the-go charging in accordance with aspects of the present disclosure. The operations of the method 1000 may be implemented by a wearable device or its components as described herein. For example, the operations of the method 1000 may be performed by a wearable device as described with reference to FIGS. 1 through 9. In some examples, a wearable device may execute a set of instructions to control the functional elements of the wearable device to perform the described functions. Additionally, or alternatively, the wearable device may perform aspects of the described functions using special-purpose hardware.

At 1005, the method may include transmitting light through one or more apertures of an inner surface of the wearable device via one or more light-emitting components of the wearable device, wherein the light is transmitted during a first time interval that the wearable device is being worn by a user. The operations of block 1005 may be performed in accordance with examples as disclosed herein. In some examples, aspects of the operations of 1005 may be performed by a sensor component 835 as described with reference to FIG. 8.

At 1010, the method may include receiving the light through the one or more apertures via one or more photodetectors during the first time interval. The operations of block 1010 may be performed in accordance with examples as disclosed herein. In some examples, aspects of the operations of 1010 may be performed by a sensor component 835 as described with reference to FIG. 8.

At 1015, the method may include generating physiological data associated with the user based at least in part on the light received by the one or more photodetectors during the first time interval. The operations of block 1015 may be performed in accordance with examples as disclosed herein. In some examples, aspects of the operations of 1015 may be performed by a sensor component 835 as described with reference to FIG. 8.

At 1020, the method may include wirelessly transferring energy from an external power source to a battery of the wearable device through an outer surface of the wearable device during the first time interval that the wearable device is being worn by the user, wherein the energy is wirelessly transferred using an inductive charging procedure, a contact-based charging procedure, or both. The operations of block 1020 may be performed in accordance with examples as disclosed herein. In some examples, aspects of the operations of 1020 may be performed by a charging component 840 as described with reference to FIG. 8.

It should be noted that the methods described above describe possible implementations, and that the operations and the steps may be rearranged or otherwise modified and that other implementations are possible. Furthermore, aspects from two or more of the methods may be combined.

A method is described. The method may include a housing comprising an inner surface and an outer surface that is opposite the inner surface, wherein the inner surface is configured to contact a tissue of a user while the wearable device is being worn by the user, a battery positioned at least partially within the housing, one or more sensors positioned at least partially within the housing and electrically coupled with the battery, the one or more sensors configured to acquire physiological data from the user through one or more apertures in the inner surface while the wearable device is being worn by the user, and a wireless charging component disposed at least partially within the housing, wherein the wireless charging component is configured to perform an inductive charging procedure, a contact-based charging procedure, or both, to wirelessly transfer energy from an external power source to the battery through the outer surface of the wearable device while the wearable device is being worn by the user.

An apparatus is described. The apparatus may include at least one processor, at least one memory coupled with the at least one processor, and instructions stored in the at least one memory. The instructions may be executable by the at least one processor to cause the apparatus to a housing comprise an inner surface and an outer surface that is opposite the inner surface, wherein the inner surface is configured to contact a tissue of a user while the wearable device is being worn by the user, a battery position at least partially within the housing, one or more sensors position at least partially within the housing and electrically coupled with the battery, the one or more sensors configured to acquire physiological data from the user through one or more apertures in the inner surface while the wearable device is being worn by the user, and a wireless charge component disposed at least partially within the housing, wherein the wireless charging component is configured to perform an inductive charging procedure, a contact-based charging procedure, or both, to wirelessly transfer energy from an external power source to the battery through the outer surface of the wearable device while the wearable device is being worn by the user.

Another apparatus is described. The apparatus may include means for a housing comprising an inner surface and an outer surface that is opposite the inner surface, wherein the inner surface is configured to contact a tissue of a user while the wearable device is being worn by the user, means for a battery positioned at least partially within the housing, means for one or more sensors positioned at least partially within the housing and electrically coupled with the battery, the one or more sensors configured to acquire physiological data from the user through one or more apertures in the inner surface while the wearable device is being worn by the user, and means for a wireless charging component disposed at least partially within the housing, wherein the wireless charging component is configured to perform an inductive charging procedure, a contact-based charging procedure, or both, to wirelessly transfer energy from an external power source to the battery through the outer surface of the wearable device while the wearable device is being worn by the user.

A non-transitory computer-readable medium storing code is described. The code may include instructions executable by a processor to a housing comprise an inner surface and an outer surface that is opposite the inner surface, wherein the inner surface is configured to contact a tissue of a user while the wearable device is being worn by the user, a battery position at least partially within the housing, one or more sensors position at least partially within the housing and electrically coupled with the battery, the one or more sensors configured to acquire physiological data from the user through one or more apertures in the inner surface while the wearable device is being worn by the user, and a wireless charge component disposed at least partially within the housing, wherein the wireless charging component is configured to perform an inductive charging procedure, a contact-based charging procedure, or both, to wirelessly transfer energy from an external power source to the battery through the outer surface of the wearable device while the wearable device is being worn by the user.

In some examples of the method, apparatuses, and non-transitory computer-readable medium described herein, the wireless charging component, an additional wireless charging component, or both, may be further configured to wirelessly transfer additional energy from a charging device to the battery through the inner surface of the wearable device while the wearable device may be positioned on or within the charging device.

Some examples of the method, apparatuses, and non-transitory computer-readable medium described herein may further include operations, features, means, or instructions for generate a first set of instructions configured to cause the one or more sensors to acquire the physiological data from the user during a first time interval and generate a second set of instructions configured to cause the wireless charging component to wirelessly transfer the energy from the external power source to the battery during the first time interval.

In some examples of the method, apparatuses, and non-transitory computer-readable medium described herein, selectively adjust at least one measurement parameter used by the one or more sensors to acquire the physiological data based at least in part on the wireless charging component being configured to wireless transfer the energy during the first time interval that the physiological data may be acquired.

In some examples of the method, apparatuses, and non-transitory computer-readable medium described herein, selectively adjusting the at least one measurement parameter comprises increasing a current or voltage applied to a light-emitting component, selectively activating at least one sensor of the one or more sensors, selectively adjusting a type of measurement performed by the one or more sensors, or any combination thereof.

Some examples of the method, apparatuses, and non-transitory computer-readable medium described herein may further include operations, features, means, or instructions for determine a charging pattern associated with the wireless charging component based at least in part on one or more prior energy transfer occasions between the external power source and the battery and selectively adjust at least one measurement parameter used by the one or more sensors to acquire the physiological data based at least in part on one or more characteristics of the charging pattern.

In some examples of the method, apparatuses, and non-transitory computer-readable medium described herein, the one or more characteristics of the charging pattern comprise a timing of energy transfer occasions between the external power source and the battery, a frequency of energy transfer occasions between the external power source and the battery, or both.

In some examples of the method, apparatuses, and non-transitory computer-readable medium described herein, the wireless charging component comprises a first inductive charging component disposed beneath or at least partially within the outer surface of the housing and the first inductive charging component may be configured to wirelessly interface with a second inductive charging component of the external power source to wirelessly transfer the energy.

In some examples of the method, apparatuses, and non-transitory computer-readable medium described herein, the wireless charging component comprises a first electrical contact component within the outer surface of the housing and the first electrical contact component may be configured to physically contact a second electrical contact component of the external power source to wirelessly transfer the energy.

In some examples of the method, apparatuses, and non-transitory computer-readable medium described herein, the wireless charging component may be configured to wirelessly transfer the energy from the external power source to the battery based at least in part on a power level of the external power source satisfying a threshold power level.

In some examples of the method, apparatuses, and non-transitory computer-readable medium described herein, the external power source may be disposed on or within a mobile phone, a case associated with the mobile phone, a steering wheel, a steering wheel cover, a coffee mug, a water bottle, a computer mouse, a keyboard, a glove, a watch wearable device, or a wearable ring device.

In some examples of the method, apparatuses, and non-transitory computer-readable medium described herein, the wearable device comprises a wearable ring device and the inner surface and the outer surface of the housing comprise an inner circumferential surface and an outer circumferential surface, respectively.

In some examples of the method, apparatuses, and non-transitory computer-readable medium described herein, the wireless charging component may be further configured to wirelessly transfer energy from the external power source to the battery through the outer surface of the wearable device while the wearable device may be not being worn by the user.

In some examples of the method, apparatuses, and non-transitory computer-readable medium described herein, one or more magnetic elements positioned at least partially within the housing, the one or more magnetic elements configured to magnetically couple with one or more second magnetic elements associated with the external power source.

In some examples of the method, apparatuses, and non-transitory computer-readable medium described herein, one or more coils positioned within the housing and coupled with the wireless charging component, the one or more coils may be configured to electrically couple with one or more second coils associated with the external power source to support wireless transfer of energy.

In some examples of the method, apparatuses, and non-transitory computer-readable medium described herein, the one or more coils wrap around an inner circumferential surface of the housing.

A method for charging a wearable device is described. The method may include transmitting light through one or more apertures of an inner surface of the wearable device via one or more light-emitting components of the wearable device, wherein the light is transmitted during a first time interval that the wearable device is being worn by a user, receiving the light through the one or more apertures via one or more photodetectors during the first time interval, generating physiological data associated with the user based at least in part on the light received by the one or more photodetectors during the first time interval, and wirelessly transferring energy from an external power source to a battery of the wearable device through an outer surface of the wearable device during the first time interval that the wearable device is being worn by the user, wherein the energy is wirelessly transferred using an inductive charging procedure, a contact-based charging procedure, or both.

An apparatus for charging a wearable device is described. The apparatus may include at least one processor, at least one memory coupled with the at least one processor, and instructions stored in the at least one memory. The instructions may be executable by the at least one processor to cause the apparatus to transmit light through one or more apertures of an inner surface of the wearable device via one or more light-emitting components of the wearable device, wherein the light is transmitted during a first time interval that the wearable device is being worn by a user, receive the light through the one or more apertures via one or more photodetectors during the first time interval, generate physiological data associated with the user based at least in part on the light received by the one or more photodetectors during the first time interval, and wirelessly transfer energy from an external power source to a battery of the wearable device through an outer surface of the wearable device during the first time interval that the wearable device is being worn by the user, wherein the energy is wirelessly transferred using an inductive charging procedure, a contact-based charging procedure, or both.

Another apparatus for charging a wearable device is described. The apparatus may include means for transmitting light through one or more apertures of an inner surface of the wearable device via one or more light-emitting components of the wearable device, wherein the light is transmitted during a first time interval that the wearable device is being worn by a user, means for receiving the light through the one or more apertures via one or more photodetectors during the first time interval, means for generating physiological data associated with the user based at least in part on the light received by the one or more photodetectors during the first time interval, and means for wirelessly transferring energy from an external power source to a battery of the wearable device through an outer surface of the wearable device during the first time interval that the wearable device is being worn by the user, wherein the energy is wirelessly transferred using an inductive charging procedure, a contact-based charging procedure, or both.

A non-transitory computer-readable medium storing code for charging a wearable device is described. The code may include instructions executable by a processor to transmit light through one or more apertures of an inner surface of the wearable device via one or more light-emitting components of the wearable device, wherein the light is transmitted during a first time interval that the wearable device is being worn by a user, receive the light through the one or more apertures via one or more photodetectors during the first time interval, generate physiological data associated with the user based at least in part on the light received by the one or more photodetectors during the first time interval, and wirelessly transfer energy from an external power source to a battery of the wearable device through an outer surface of the wearable device during the first time interval that the wearable device is being worn by the user, wherein the energy is wirelessly transferred using an inductive charging procedure, a contact-based charging procedure, or both.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable ROM (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A wearable device, comprising:
a housing comprising an inner surface and an outer surface that is opposite the inner surface, wherein the inner surface is configured to contact a tissue of a user while the wearable device is being worn by the user;
a battery positioned at least partially within the housing;
one or more sensors positioned at least partially within the housing and electrically coupled with the battery, the one or more sensors configured to acquire physiological data from the user through one or more apertures in the inner surface while the wearable device is being worn by the user; and
a wireless charging component disposed at least partially within the housing, wherein the wireless charging component is configured to perform an inductive charging procedure, a contact-based charging procedure, a photovoltaic charging procedure, or any combination thereof, to wirelessly transfer energy from an external power source to the battery through the outer surface of the wearable device, the inner surface of the wearable device, or both, while the wearable device is being worn by the user.

2. The wearable device of claim 1, wherein the wireless charging component, an additional wireless charging component, or both, is further configured to wirelessly transfer additional energy from a charging device to the battery through the inner surface of the wearable device while the wearable device is positioned on or within the charging device.

3. The wearable device of claim 1, further comprising one or more processors communicatively coupled with the one or more sensors and the wireless charging component, wherein the one or more processors are configured to:
generate a first set of instructions configured to cause the one or more sensors to acquire the physiological data from the user during a first time interval; and
generate a second set of instructions configured to cause the wireless charging component to wirelessly transfer the energy from the external power source to the battery during the first time interval.

4. The wearable device of claim 3, wherein the one or more processors are further configured to selectively adjust at least one measurement parameter used by the one or more sensors to acquire the physiological data based at least in part on the wireless charging component being configured to wireless transfer the energy during the first time interval that the physiological data is acquired.

5. The wearable device of claim 4, wherein selectively adjusting the at least one measurement parameter comprises increasing a current or voltage applied to a light-emitting component, selectively activating at least one sensor of the one or more sensors, selectively adjusting a type of measurement performed by the one or more sensors, or any combination thereof.

6. The wearable device of claim 1, further comprising one or more processors communicatively coupled with the one or more sensors and the wireless charging component, wherein the one or more processors are configured to:
determine a charging pattern associated with the wireless charging component based at least in part on one or more prior energy transfer occasions between the external power source and the battery; and
selectively adjust at least one measurement parameter used by the one or more sensors to acquire the physiological data based at least in part on one or more characteristics of the charging pattern.

7. The wearable device of claim 6, wherein the one or more characteristics of the charging pattern comprise a timing of energy transfer occasions between the external power source and the battery, a frequency of energy transfer occasions between the external power source and the battery, or both.

8. The wearable device of claim 1, wherein the wireless charging component comprises a first inductive charging component disposed beneath or at least partially within the outer surface of the housing, and wherein the first inductive charging component is configured to wirelessly interface with a second inductive charging component of the external power source to wirelessly transfer the energy.

9. The wearable device of claim 1, wherein the wireless charging component comprises a first electrical contact component within the outer surface of the housing, and wherein the first electrical contact component is configured to physically contact a second electrical contact component of the external power source to wirelessly transfer the energy.

10. The wearable device of claim 1, wherein the wireless charging component is configured to wirelessly transfer the energy from the external power source to the battery based at least in part on a power level of the external power source satisfying a threshold power level.

11. The wearable device of claim 1, wherein the external power source is disposed on or within a mobile phone, a case associated with the mobile phone, a steering wheel, a steering wheel cover, a coffee mug, a water bottle, a computer mouse, a keyboard, a glove, a watch wearable device, or a wearable ring device.

12. The wearable device of claim 1, wherein the wearable device comprises a wearable ring device, and wherein the inner surface and the outer surface of the housing comprise an inner circumferential surface and an outer circumferential surface, respectively.

13. The wearable device of claim 1, wherein the wireless charging component is further configured to wirelessly transfer energy from the external power source to the battery through the outer surface of the wearable device while the wearable device is not being worn by the user.

14. The wearable device of claim 1, further comprising:
one or more magnetic elements positioned at least partially within the housing, the one or more magnetic elements configured to magnetically couple with one or more additional magnetic elements associated with the external power source.

15. The wearable device of claim 1, further comprising:
one or more charging coils positioned within the housing and coupled with the wireless charging component, the one or more charging coils configured to electrically couple with one or more additional charging coils associated with the external power source to perform the inductive charging procedure.

16. The wearable device of claim 15, wherein the one or more charging coils wrap around an inner circumferential surface of the housing.

17. The wearable device of claim 1, wherein the wearable device comprises a wearable ring device, and wherein the external power source comprises a charging ring device that is configured to perform the inductive charging procedure, the contact-based charging procedure, the photovoltaic charging procedure, or any combination thereof, when the wearable ring device and the charging ring device are worn on a same finger of the user.

18. The wearable device of claim 17, wherein the wireless charging component is disposed proximate to the inner surface of the wearable ring device, and wherein an additional wireless charging component of the charging ring device is positioned between the inner surface of the wearable ring device and the tissue of the user during the inductive charging procedure and while the wearable ring device and the charging ring device are worn by the user.

19. A method for charging a wearable device, comprising:
transmitting light through one or more apertures of an inner surface of the wearable device via one or more light-emitting components of the wearable device, wherein the light is transmitted during a first time interval that the wearable device is being worn by a user;
receiving the light through the one or more apertures via one or more photodetectors during the first time interval;
generating physiological data associated with the user based at least in part on the light received by the one or more photodetectors during the first time interval; and
wirelessly transferring energy from an external power source to a battery of the wearable device through an outer surface of the wearable device during the first time interval that the wearable device is being worn by the user, wherein the energy is wirelessly transferred using an inductive charging procedure, a contact-based charging procedure, a photovoltaic charging procedure, or any combination thereof.
